(19) United States
(12) Patent Application Publication
(10) Pub. No.: US 2026/0165025 A1
(43) Pub. Date: Jun. 11, 2026

(54) ORGANIC SEMICONDUCTOR MATERIAL AND ORGANIC OPTOELECTRONIC DEVICE USING THE SAME

(71) Applicant: Raynergy Tek Incorporation, Hsinchu (TW)

(72) Inventors: Cheng-Chang Lai, Hsinchu (TW); Yu-Tang Hsiao, Hsinchu (TW)

(21) Appl. No.: 19/409,826

(22) Filed: Dec. 5, 2025

Related U.S. Application Data
(60) Provisional application No. 63/730,467, filed on Dec. 11, 2024.

(51) Int. Cl. H10K 85/60; C07D 495/22; H10K 30/40; H10K 50/11

(52) U.S. Cl. CPC H10K 85/657; C07D 495/22; H10K 85/615; H10K 30/40; H10K 50/11

(57) ABSTRACT

An organic semiconductor material comprising a structure of Formula I:

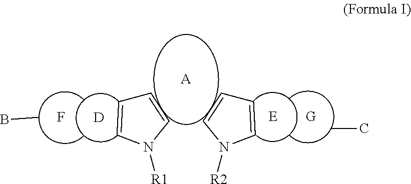

The organic semiconductor material has an optical bandgap lower than 1.15 eV, and exhibits good solubility and thermal stability. The present invention also provides an organic optoelectronic device comprising a first electrode, an active layer, and a second electrode. The active layer contains the organic semiconductor material. The organic optoelectronic device is applied to a short-wave infrared (SWIR) organic photodetector, and is fabricated using non-halogenated solvents. The organic optoelectronic device exhibits a low dark current density, excellent responsivity and detectivity in the short-wave infrared region, as well as good thermal stability.

The present application is based on, and claims priority from, America provisional patent application number U.S. 63/730,467 filed on 2024 Dec. 11, and the disclosure of which is hereby incorporated by reference herein in its entirety.

### BACKGROUND OF THE INVENTION

### Field of the Invention

The present invention relates to an organic material applied in an organic optoelectronic device, and in particular to an organic semiconductor material, an organic composition comprising the organic semiconductor material, and an organic optoelectronic device.

### Description of the Prior Art

Compared to traditional inorganic optoelectronic devices, organic optoelectronic devices have wide absorption wavelength range, high absorption coefficient, and adjustable structures, and their light absorption range, energy level and solubility can be adjusted according to the target requirements. In addition, organic materials have the advantages of low cost, flexibility, low toxicity and large-area production of devices, so that organic optoelectronic devices have good competitiveness in various fields, such as organic field effect transistors (OFETs), organic light emitting diodes (OLEDs), organic photovoltaics (OPVs) and organic photodetectors (OPDs).

An organic photodetector is a semiconductor device that converts optical signals into electrical signals. Within its operational wavelength range, the organic photodetector should exhibit high sensitivity, high response rate, minimal noise, small sensing area, low operating voltage, and high reliability. Organic photovoltaic cells, on the other hand, are used for generating electricity from sunlight, and the two devices share certain similarities in structure and material applications. However, their primary difference lies in their intended functions: an organic photovoltaic cell is a power-generating device, and its light source is sunlight; therefore, its design primarily focuses on photoelectric conversion efficiency. In contrast, an organic photodetector is a device that senses light and generates electrical signals, and its light source is typically an emission source of a specific wavelength range. The light intensity is much weaker than sunlight, resulting in a significantly lower electrical output signal. Consequently, the device design of organic photodetectors requires more stringent noise-suppression considerations.

The performance of an organic photodetector is primarily determined by its detectivity, which is influenced by its responsivity and dark current, and is therefore different from the design focus of organic photovoltaic cells. In addition, another important characteristic of organic photodetectors is wavelength selectivity. To avoid background interference, they are typically designed to operate in the near-infrared to short-wavelength infrared regions. Currently reported organic materials in the literature exhibit major absorption bands concentrated around 900 nm, with an optical bandgap of approximately 1.3 eV, thereby limiting their application to the near-infrared region. Moreover, most existing device fabrication processes rely on halogenated solvents, which are less environmentally friendly and constrain their practical application and industrial development.

In view of the foregoing, it is an important issue to develop an organic semiconductor material having an optical bandgap significantly lower than 1.3 eV, such that it may be applied to organic photodetectors operating in the short-wavelength infrared region. In other words, it is highly desirable for the organic photodetector to exhibit a low dark current density, good responsivity and detectivity in the short-wavelength infrared region, and excellent thermal stability of the device.

### SUMMARY OF THE INVENTION

In view of this, one category of the present invention is to provide an organic semiconductor material comprising a structure of Formula I:

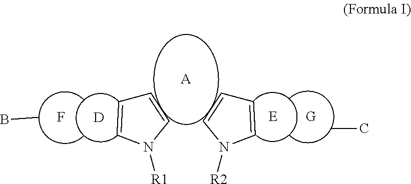

Wherein, A is a monocyclic ring or polycyclic ring, and the polycyclic ring comprises at least two six-membered rings. B and C are polycyclic rings, each comprising at least one six-membered ring. D, E, F, and G are monocyclic rings or polycyclic rings, each comprising at least one five-membered ring. R1 and R2 are independently selected from the following groups and any combinations thereof: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl.

Wherein, A is a C6-C30 monocyclic ring or polycyclic ring, and the polycyclic ring comprises at least two six-membered rings. B and C are C3-C20 aryl with substituents or heteroaryl with substituents, wherein the substituents comprise at least one ketone group. D, E, F, and G are a C4-C30 monocyclic ring or polycyclic ring.

Wherein, an optical bandgap of the organic semiconductor material is less than 1.15 eV.

Wherein, A is selected from one of the following structures, in which the dashed line represents an outward bonding position:

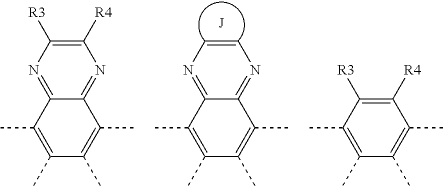

Wherein, R3 and R4 are the same or different and are independently selected from the following groups and any combinations thereof: hydrogen, deuterium, tritium, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl. J is selected from the following structures, in which the dashed line represents an outward bonding position:

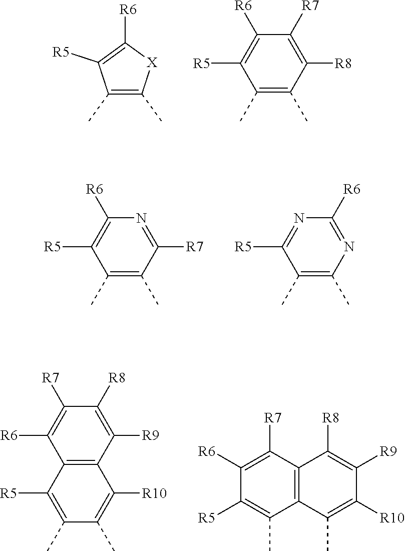

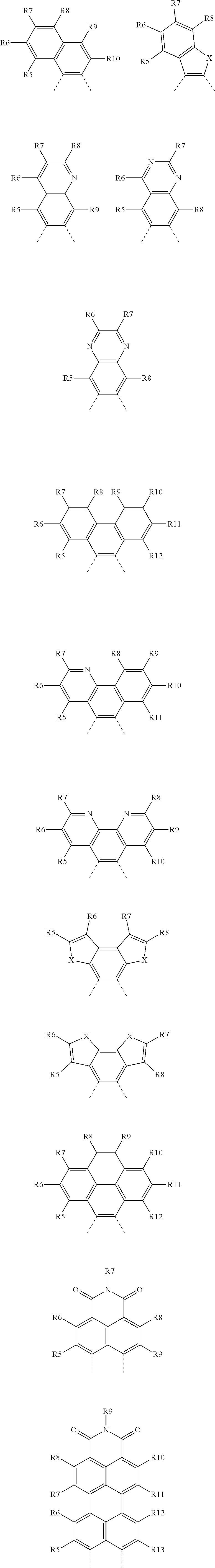

Wherein, X is selected from O, S, Se, or N—R14. R5, R6, R7, R8, R9, R10, R11, R12, R13, and R14 are independently selected from the following groups and any combinations thereof: hydrogen, deuterium, tritium, halogen, cyano, C1-C30 alkyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl.

Wherein, B and C are independently selected from the following structures, in which the dashed line represents an outward bonding position:

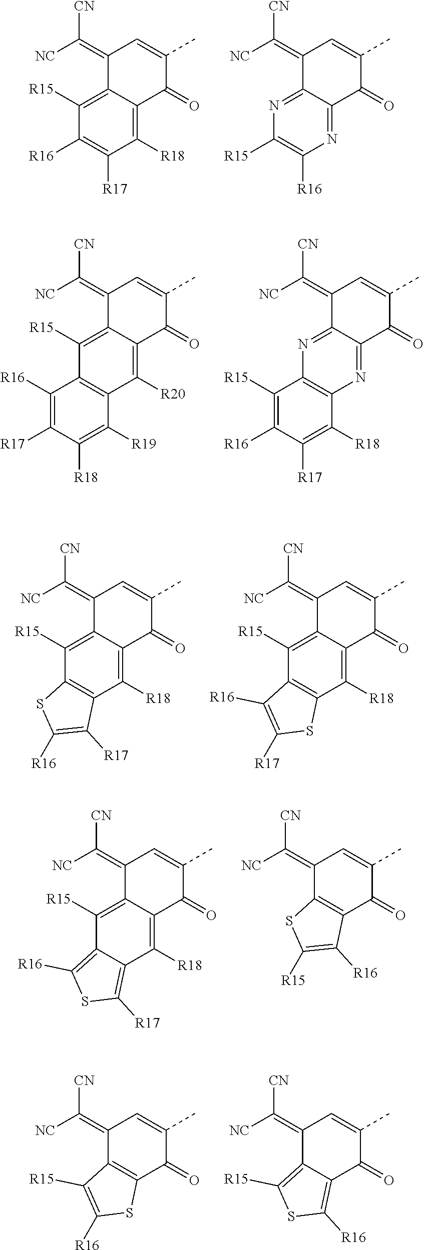

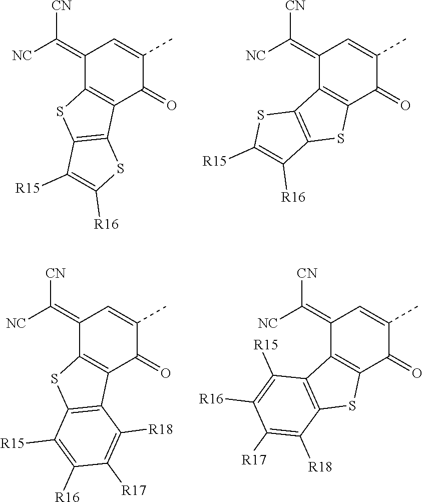

Wherein, R15, R16, R17, R18, R19, and R20 are independently selected from the following groups and any combinations thereof: C1-C30 alkyl, C1-C30 silyl, C1-C30 alkoxy, C1-C30 alkylthio, C1-C30 haloalkyl, halogen, hydrogen, deuterium, tritium, and cyano.

Wherein, D and E are independently selected from the following structures, in which the dashed line represents an outward bonding position:

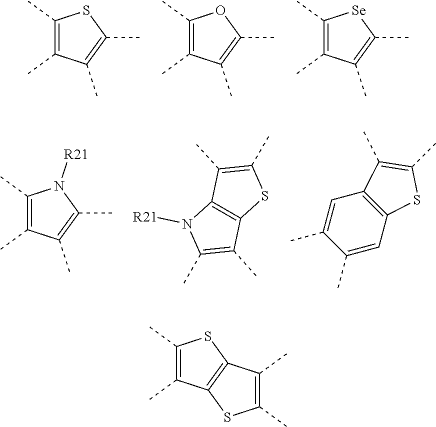

Wherein, R21 is selected from the following groups and any combinations thereof: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl.

Wherein, F and G are independently selected from the following structures, in which the dashed line represents an outward bonding position:

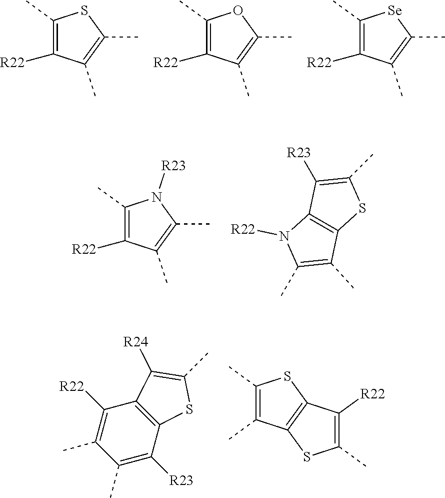

Wherein, R22, R23, and R24 are independently selected from the following groups and any combinations thereof: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, C2-C30 esteryl heteroaryl, and hydrogen.

The second category of the present invention is to provide an organic composition comprising at least one P-type organic semiconductor material and at least one N-type organic semiconductor material. The P-type organic semiconductor material comprises at least one organic conjugated polymer or one organic conjugated small molecule. The N-type organic semiconductor material comprises at least one organic semiconductor material described previously.

Wherein, the P-type organic semiconductor material is further selected from at least one organic conjugated polymer, and the organic conjugated polymer is formed from a plurality of monomer units, the monomer units being selected from the following structures and combinations thereof:

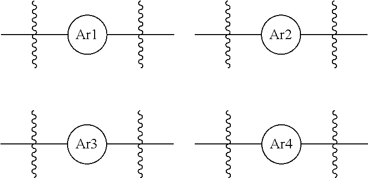

Wherein, Ar1, Ar2, Ar3, and Ar4 are independently selected from a monocyclic ring or a polycyclic ring.

Wherein, the organic conjugated polymer further comprises the following structures and any combinations thereof:

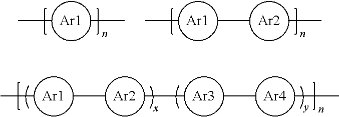

Wherein, Ar1, Ar2, Ar3, and Ar4 are monocyclic rings or polycyclic rings each comprising C3-C30 ring atoms. n is the number of repeating units, and n is a positive integer from 1 to 1000. x and y are mole fractions, wherein 0<x<1, 0<y<1, and x+y=1.

Wherein, Ar1 and Ar3 are independently selected from the following structures:

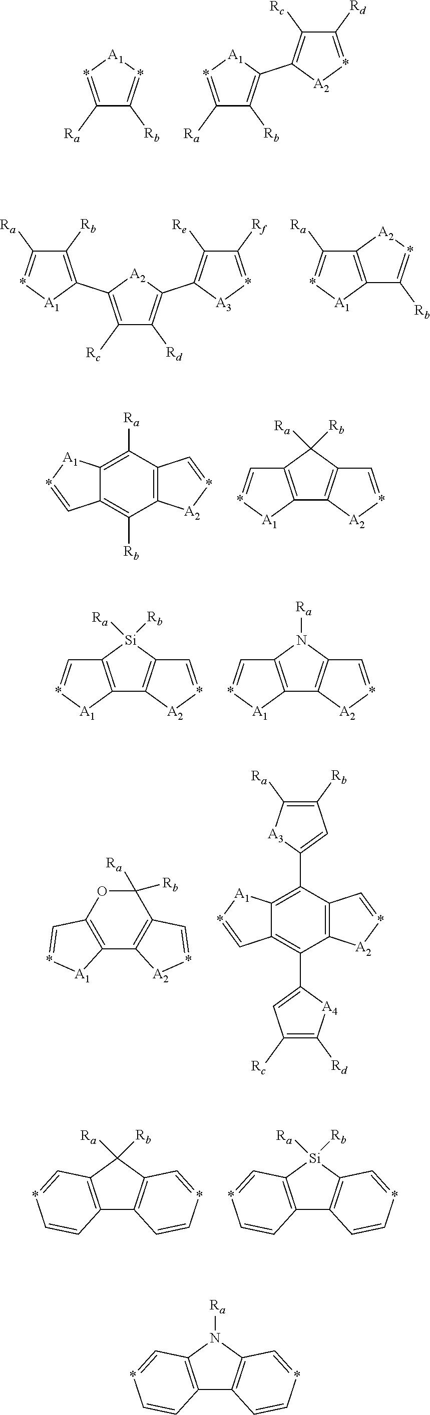

Wherein, A₁, A₂, A₃, and A₄ are independently selected from O, S, and Se. Rₐ, R_{b}, R_{c}, R_{d}, Rₑ, and R_{f} are independently selected from the following groups and any combinations thereof: hydrogen, halogen, cyano, C1-C30 alkyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl. * represents a single bond outward bonding.

Wherein, Ar2 and Ar4 are independently selected from the following structures:

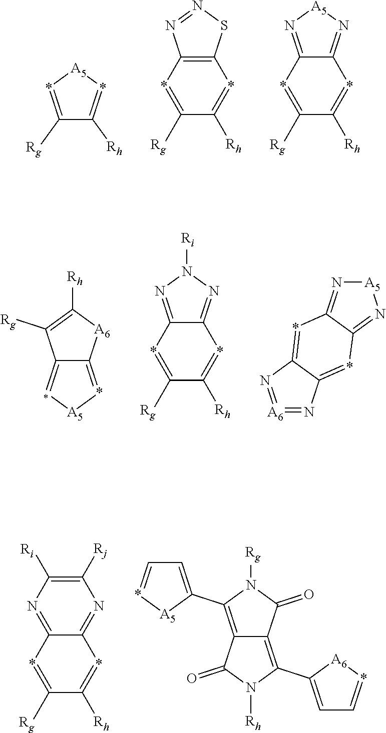

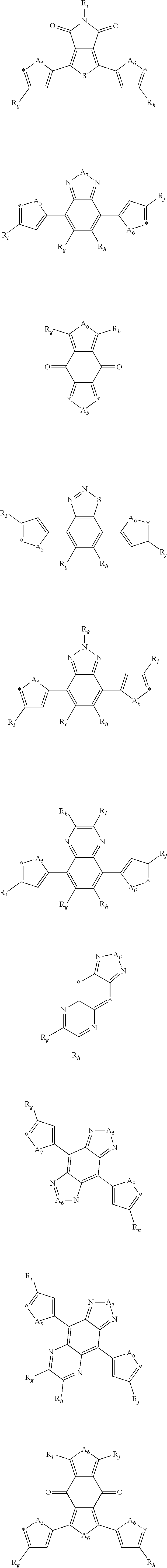

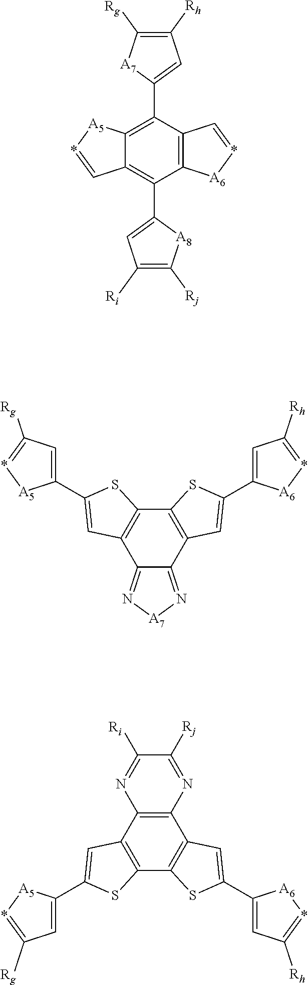

Wherein, A₅, A₆, A₇, and A₈ are independently selected from O, S, and Se. R_{g}, Rₕ, Rᵢ, Rⱼ, Rₖ, and Rₗ are independently selected from the following groups and any combinations thereof: hydrogen, halogen, cyano, C1-C30 alkyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl. * represents a single bond outward bonding.

The third category of the present invention is to provide an organic optoelectronic device comprising a first electrode, an active layer and a second electrode. The active layer at least comprises the organic semiconductor material described previously. Wherein, the active layer is disposed between the first electrode and the second electrode, and at least one of the first electrode and the second electrode is a transparent or semi-transparent electrode.

The fourth category of the present invention is to provide an organic optoelectronic device comprising a first electrode, an active layer and a second electrode. The active layer at least comprises the organic composition described previously. Wherein, the active layer is disposed between the first electrode and the second electrode, and at least one of the first electrode and the second electrode is a transparent or semi-transparent electrode.

Compared with the prior art, the organic semiconductor material of the present invention has an optical bandgap less than 1.15 eV and exhibits good solubility and thermal stability. In addition, the organic optoelectronic device of the present invention is an organic photodetector applied to the short-wavelength infrared region and is fabricated using non-halogenated solvents. The organic optoelectronic device of the present invention exhibits a low dark current density, good responsivity and detectivity in the short-wavelength infrared region, and excellent thermal stability.

### BRIEF DESCRIPTION OF THE APPENDED DRAWINGS

Some of the embodiments will be described in detail, with reference to the following figures, wherein like designations denote like members, wherein:

FIG. 1 shows a schematic structural diagram of one embodiment of an organic optoelectronic device of the present invention.

FIG. 2 shows absorption spectra in thin-film state of Comparative Example Y-QC4F and Example 1 to Example 4 of organic semiconductor materials.

FIG. 3 shows absorption spectra in thin-film state of Comparative Example Y-QC4F and Example 5 to Example 9 of organic semiconductor materials.

FIG. 4 shows thermal stability variations of Comparative Example Y-QC4F and Example 1 to Example 9 of organic semiconductor materials under a baking condition of 120° C.

FIG. 5 shows detectivity versus wavelength characteristics of Comparative Entry C, Entry 1 and Entry 2 of organic optoelectronic devices under a bias voltage of −2 V.

FIG. 6 shows dark current density-voltage characteristics of Comparative Entry C and Entry 1 to Entry 4 of organic optoelectronic devices under different bias voltages.

### DETAILED DESCRIPTION OF THE INVENTION

In order to make the advantages, spirit and features of the present invention easier and clearer, it will be detailed and discussed in the following with reference to the embodiments and the accompanying drawings. It is worth noting that the specific embodiments are merely representatives of the embodiments of the present invention, but it can be implemented in many different forms and is not limited to the embodiments described in this specification. Rather, these embodiments are provided so that this disclosure will be thorough and complete.

The terminology used in the various embodiments disclosed in the present invention is only for the purpose of describing specific embodiments, and is not intended to limit the various embodiments disclosed in the present invention. As used herein, singular forms also include plural forms unless the context clearly indicates otherwise. Unless otherwise defined, all terms (including technical and scientific terms) used in this specification have the same meanings as commonly understood by one of ordinary skill in the art to which the various embodiments disclosed herein belong. The above terms (such as those defined in commonly used dictionaries) will be interpreted as having the same meaning as the contextual meaning in the same technical field, and will not be interpreted as having an idealized or overly formal meaning, unless explicitly defined in the various embodiments disclosed herein.

In the description of this specification, the description of the reference terms “an embodiment”, “a specific embodiment” and the like means that specific features, structures, materials, or characteristics described in connection with the embodiment are included in at least one embodiment of the present invention. In this specification, the schematic expressions of the above terms do not necessarily refer to the same embodiment. Furthermore, the particular features, structures, materials, or characteristics described may be combined in any suitable manner in any one or more embodiments.

### Definition

As used herein, “donor” material and “p-type” (“P-type”) material refer to a semiconductor material, such as an organic semiconductor material, having holes as a primary current or charge carrier. In some embodiments, when a p-type semiconductor material is deposited on a substrate, it can provide the hole mobility greater than about 10⁻⁵ cm²/Vs.

As used herein, “acceptor” material and “n-type” (“N-type”) material refer to the semiconductor material, such as the organic semiconductor material, having electrons as the primary current or the charge carrier. In some embodiments, when an n-type semiconductor material is deposited on a substrate, it can provide the electron mobility of more than about 10⁻⁵ cm²/Vs.

“ [symbol] ”, “*” or dashed line in the structures listed herein represents the available bonding positions of this structure, but not limited thereto.

As used herein, “solution process” refers to a process in which a compound (e.g., a polymer), material, or composition can be used in a solution state, such as spin coating, printing (e.g., inkjet printing, gravure printing, and lithography printing), spray coating, slit coating, drop casting, dip coating, and blade coating.

As used herein, “annealing” refers to a post-deposition thermal treatment to a semi-crystalline polymer film for certain duration in the environment or under decompressed or pressurized environment. “Annealing temperature” refers to the temperature at which the polymer film or the mixed film of the polymer and other molecules can perform small-scale molecular movement and rearrangement during the annealing process. Without being limited by any particular theory, it is believed that annealing can lead to an increase in crystallinity in the polymer film and enhance the carrier mobility of the polymer film or a mixed film formed by the polymer and other molecules, and the molecules are arranged alternately to achieve the effect of independent transporting paths of effective electrons and holes.

The external quantum efficiency (EQE) as used herein is the spectral response Amp/Watt unit, which Amp is converted to the number of electrons per unit time (electron/sec) and Watt is converted to the number of photons per unit time (Photons/sec), and insert the quantum efficiency obtained by the above formula. Generally speaking, quantum efficiency (QE) refers to external quantum efficiency (EQE), also known as incident photon-electron conversion efficiency (IPCE).

Dark current density (J_{d} or J_{dark}) as used herein, also known as no-illumination current, refers to the current flows in an optoelectronic device in the absence of light irradiation.

The responsibility (R) and the detectivity (D*) as used herein are based on measuring the dark current and external quantum efficiency (EQE) of the organic photodetector, and are calculated by the following formula:

$$R ( λ ) = E Q E \frac{λ q}{hc} \qquad {D}^{*} = \frac{( λ / 1240 ) \times ( E Q E )}{\sqrt{2 {eJ}_{dark}}}$$

Wherein, λ is the wavelength, e is the elementary charge (1.602×10⁻¹⁹ Coulombs), h is Planck's constant (6.626×10⁻³⁴ m² kg/s), c is the speed of light (3×10⁸ m/sec), and J_{dark} is the dark current.

In an embodiment, an organic semiconductor material comprising a structure of Formula I:

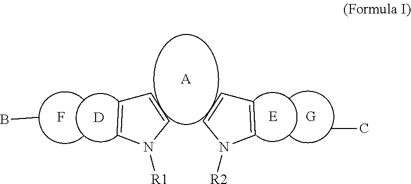

Wherein, A is a monocyclic ring or polycyclic ring with or without substituents, and the polycyclic ring comprises at least two six-membered rings. In a preferred embodiment, the monocyclic ring is a six-membered ring. B and C are polycyclic rings with or without substituents, each comprising at least one six-membered ring. D, E, F, and G are monocyclic rings or polycyclic rings with or without substituents, each comprising at least one five-membered ring. R1 and R2 are independently selected from the following groups and any combinations thereof: C1-C30 alkyl with or without substituents, C1-C30 silyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, and C2-C30 esteryl heteroaryl with or without substituents.

In practice, A is a C6-C30 monocyclic ring or polycyclic ring with or without substituents, and the polycyclic ring comprises at least two six-membered rings. B and C are C3-C20 aryl with substituents or heteroaryl with substituents, wherein the substituents comprise at least one ketone group. D, E, F, and G are a C4-C30 monocyclic ring or polycyclic ring with or without substituents.

In practice, A is selected from one of the following structures, in which the dashed line represents an outward bonding position:

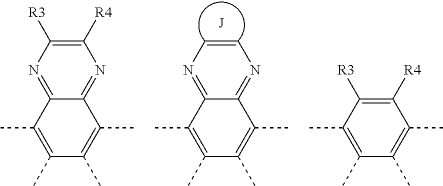

Wherein, R3 and R4 are the same or different and are independently selected from the following groups and any combinations thereof: hydrogen, deuterium, tritium, halogen, cyano, C1-C30 alkyl with or without substituents, C1-C30 silyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, and C2-C30 esteryl heteroaryl with or without substituents. J is selected from the following structures, in which the dashed line represents an outward bonding position:

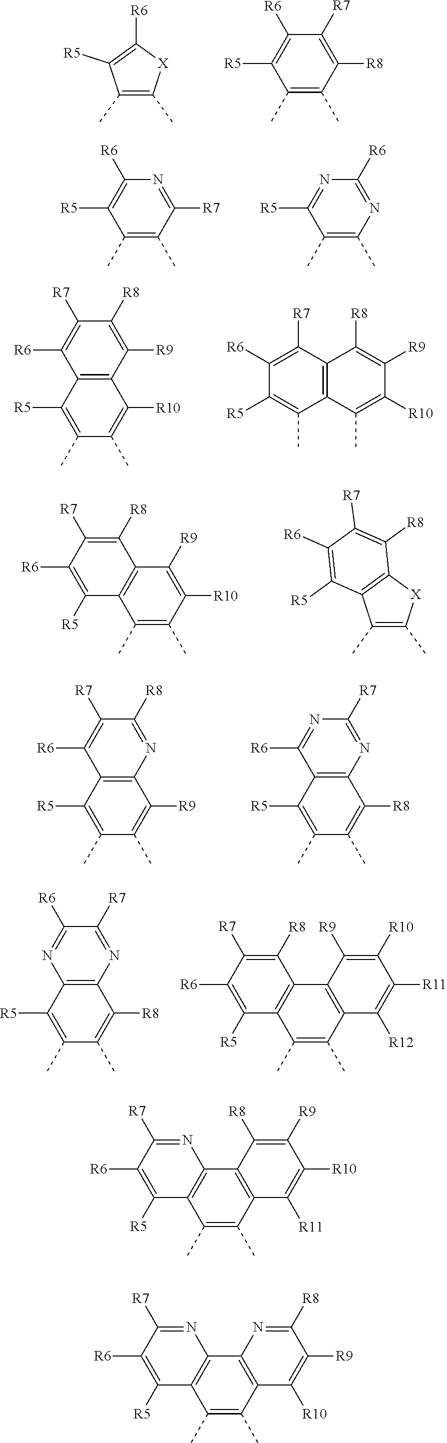

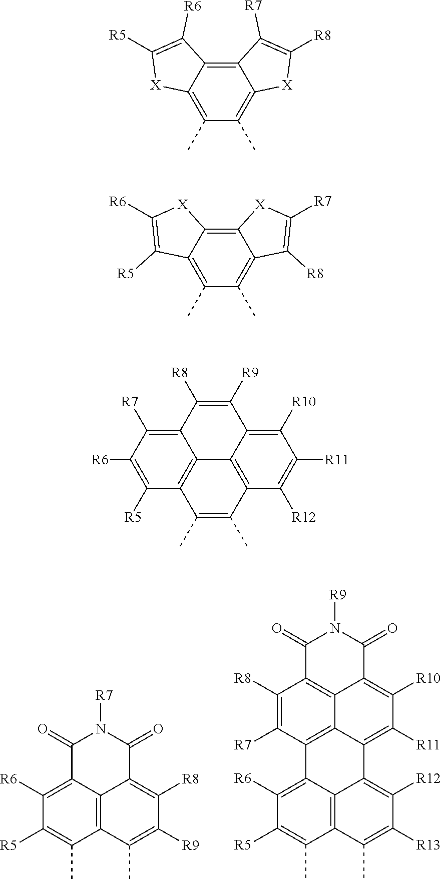

Wherein, X is selected from O, S, Se, or N—R14. R5, R6, R7, R8, R9, R10, R11, R12, R13, and R14 are independently selected from the following groups and any combinations thereof: hydrogen, deuterium, tritium, halogen, cyano, C1-C30 alkyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, and C2-C30 esteryl heteroaryl with or without substituents.

In practice, B and C are further independently selected from the following structures, in which the dashed line represents an outward bonding position:

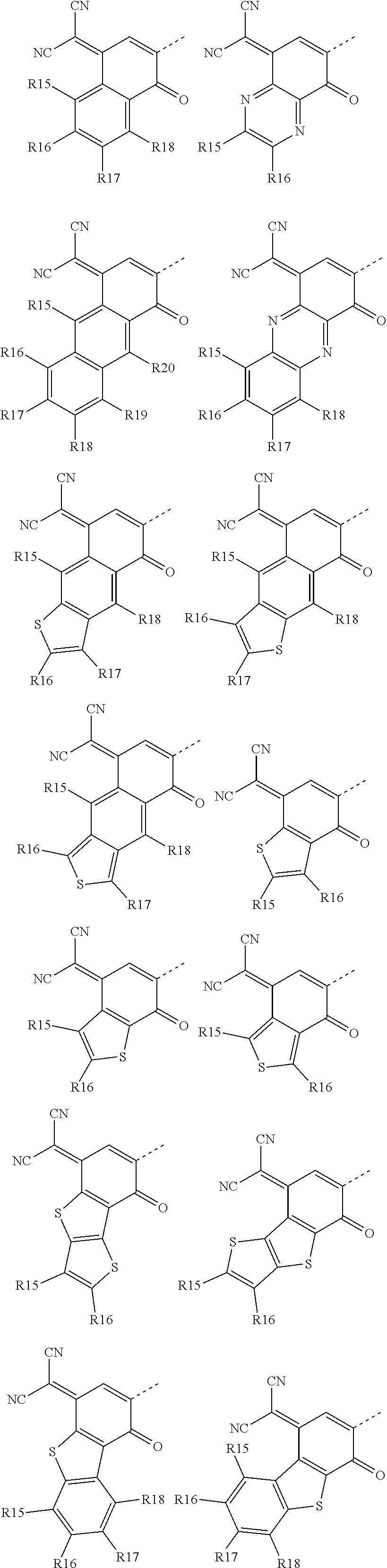

Wherein, R15, R16, R17, R18, R19, and R20 are independently selected from the following groups and any combinations thereof: C1-C30 alkyl with or without substituents, C1-C30 silyl with or without substituents, C1-C30 alkoxy with or without substituents, C1-C30 alkylthio with or without substituents, C1-C30 haloalkyl with or without substituents, halogen, hydrogen, deuterium, tritium, and cyano.

In practice, D and E are further independently selected from the following structures, in which the dashed line represents an outward bonding position:

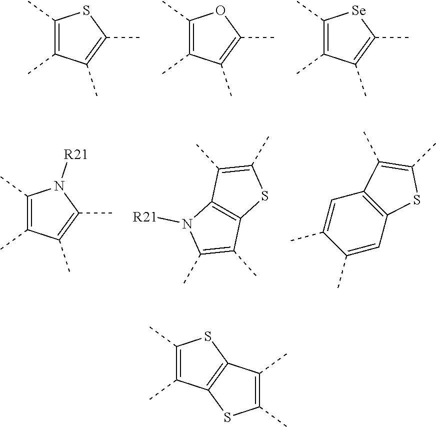

Wherein, R21 is selected from the following groups and any combinations thereof: C1-C30 alkyl with or without substituents, C1-C30 silyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, and C2-C30 esteryl heteroaryl with or without substituents.

In practice, F and G are further independently selected from the following structures, in which the dashed line represents an outward bonding position:

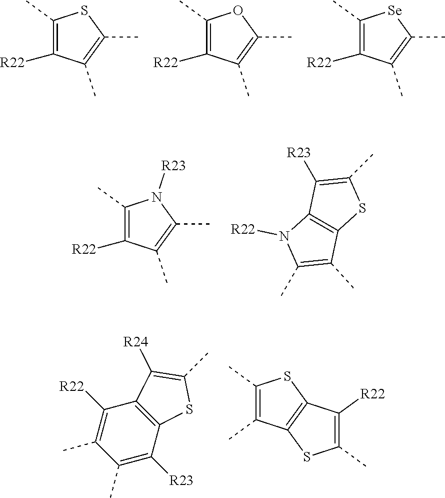

Wherein, R22, R23, and R24 are independently selected from the following groups and any combinations thereof: C1-C30 alkyl with or without substituents, C1-C30 silyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, C2-C30 esteryl heteroaryl with or without substituents, and hydrogen.

In detail, the organic semiconductor material may comprise Example 1 to Example 45 as described below:

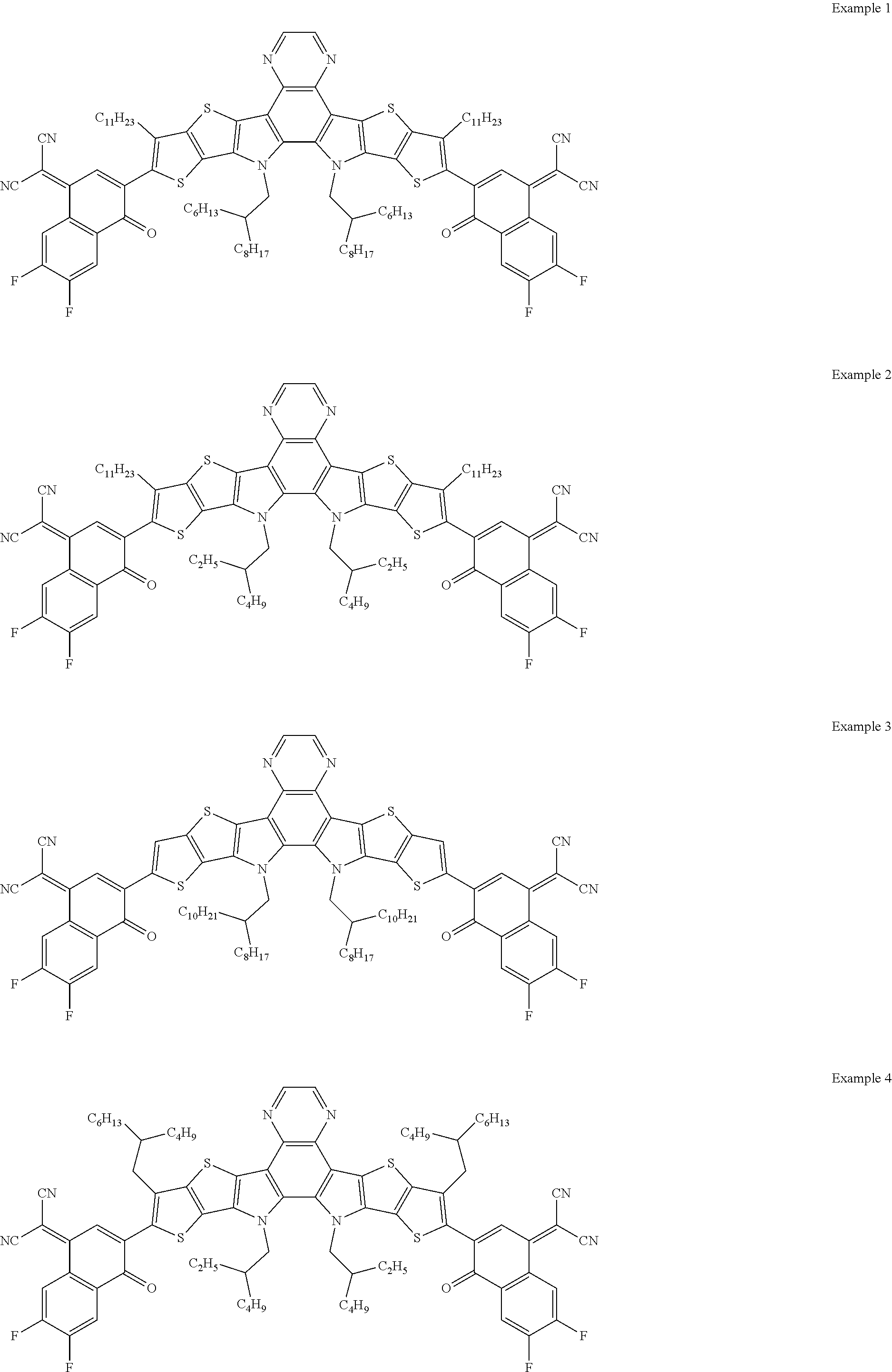

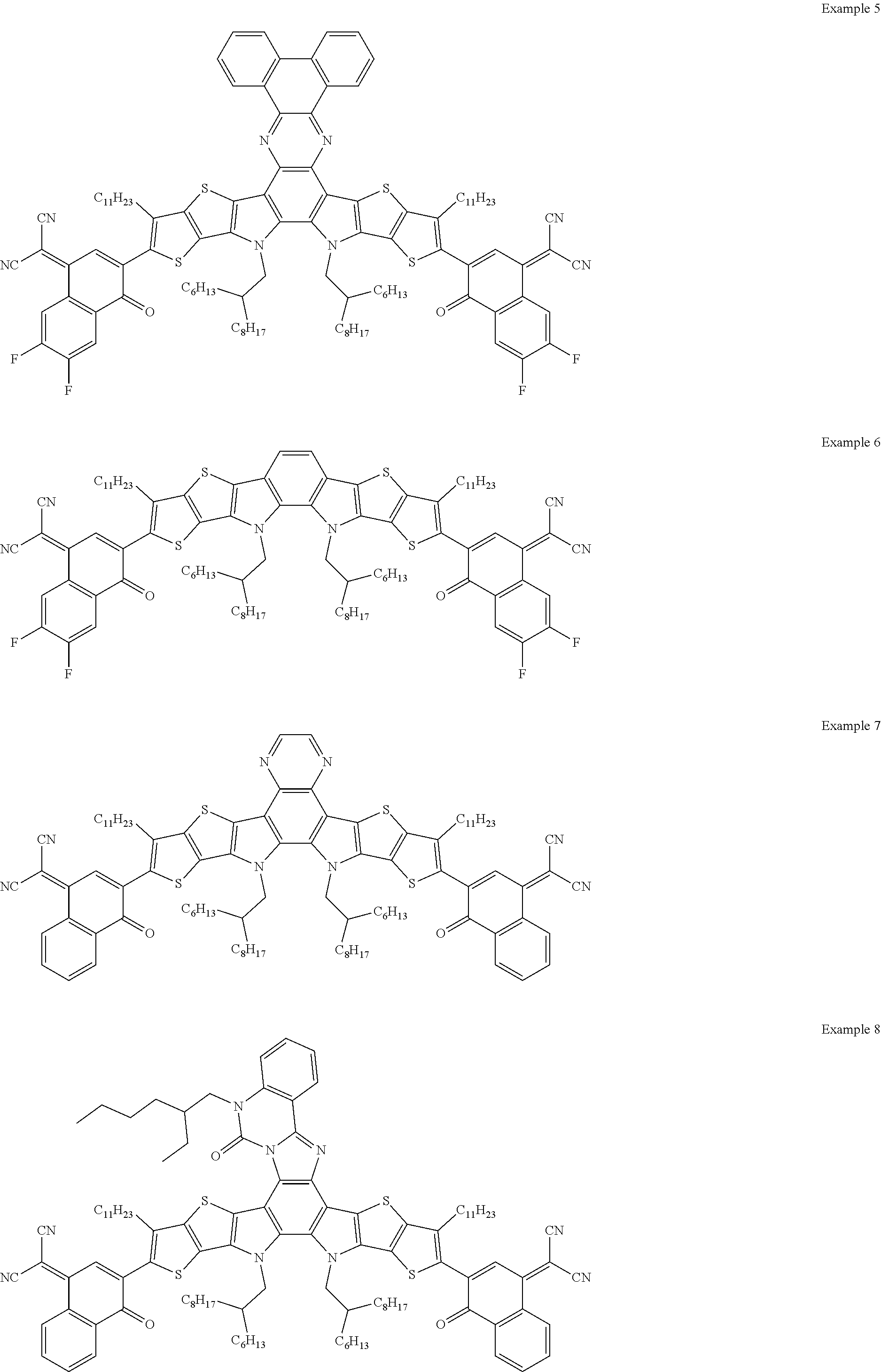

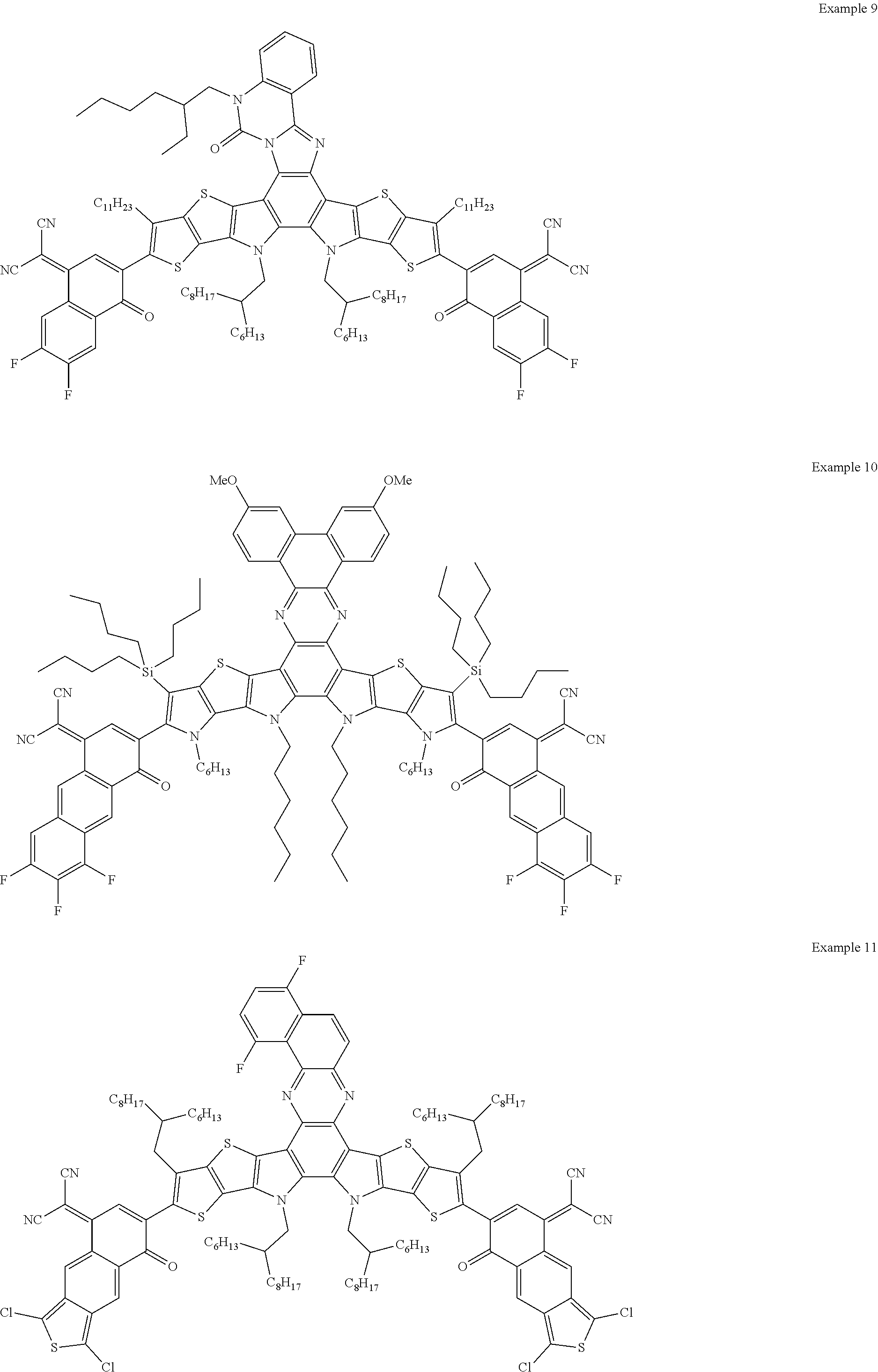

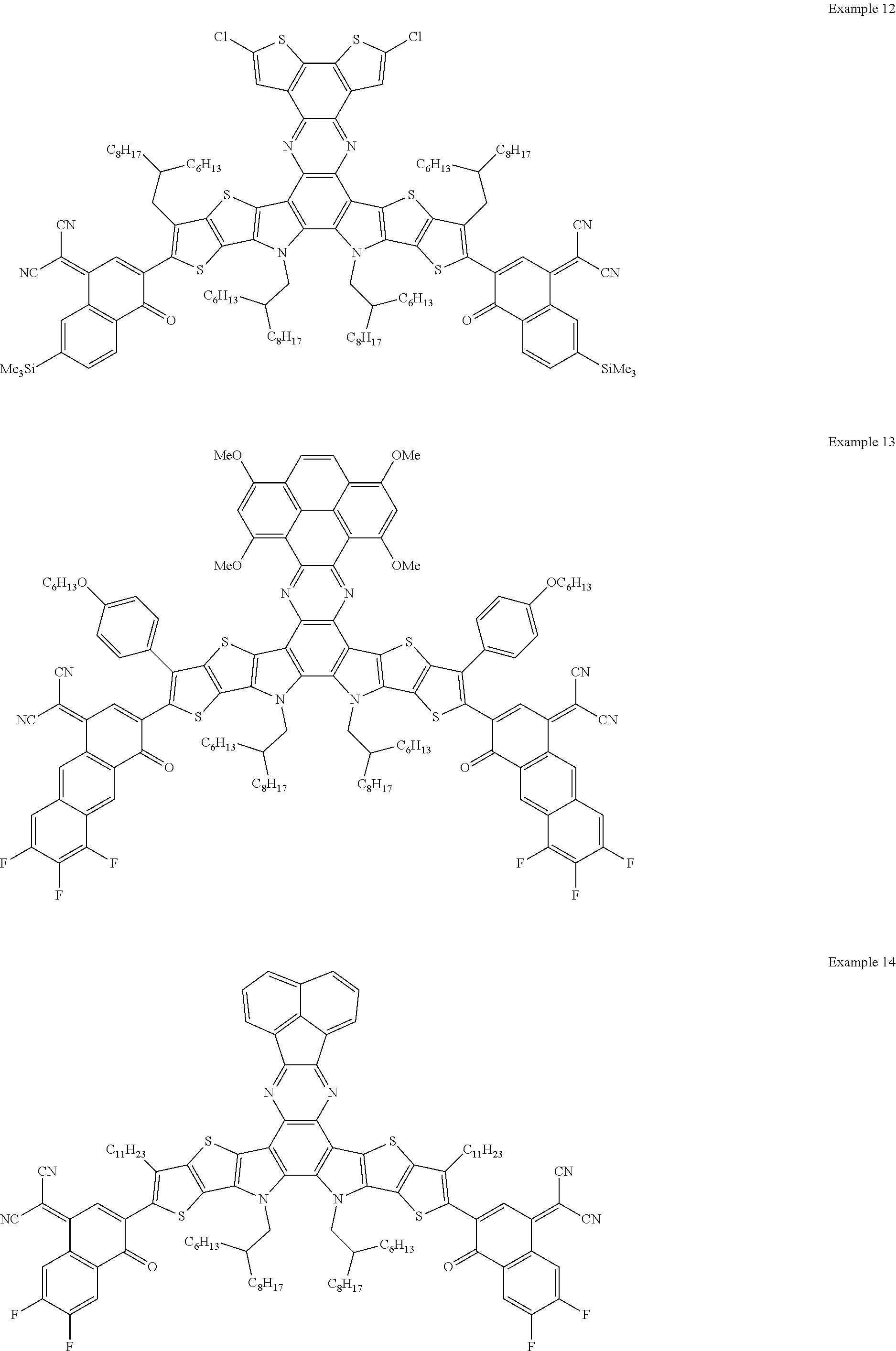

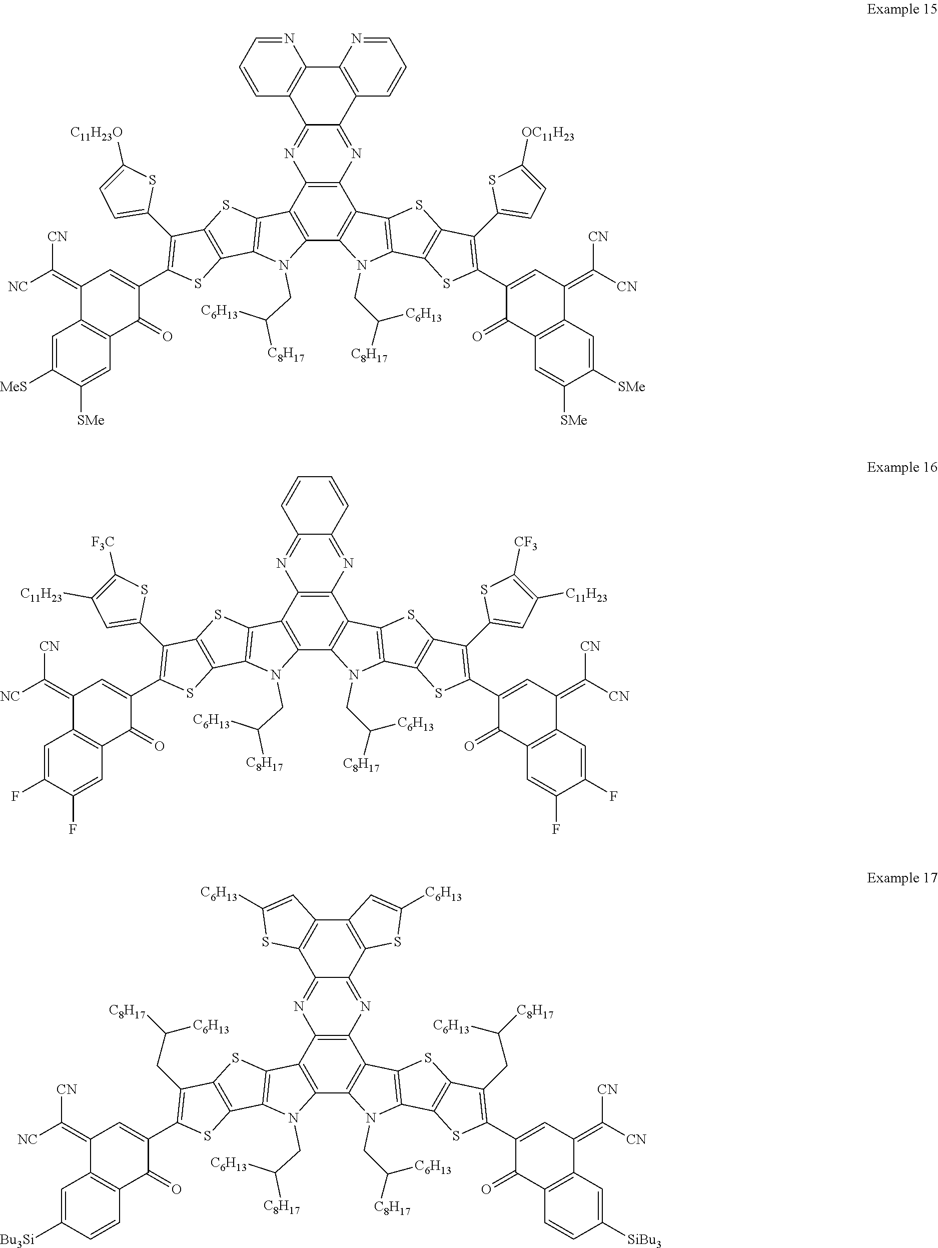

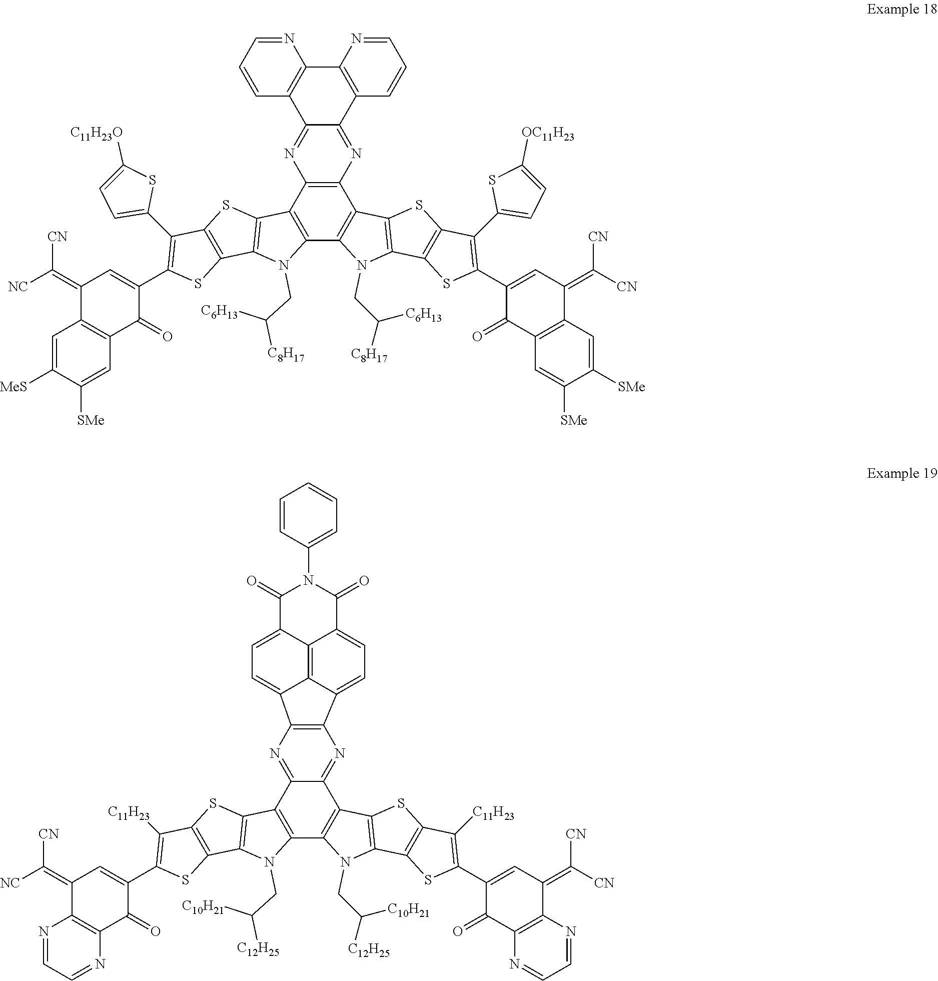

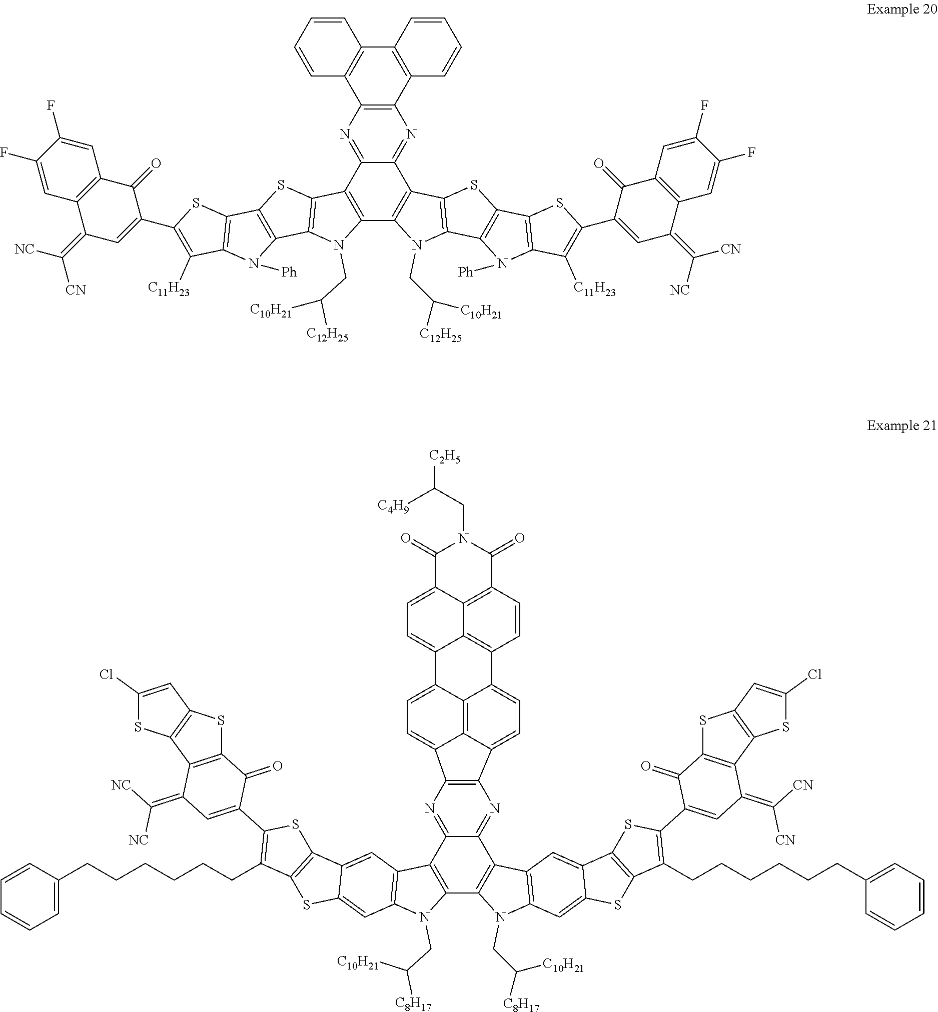

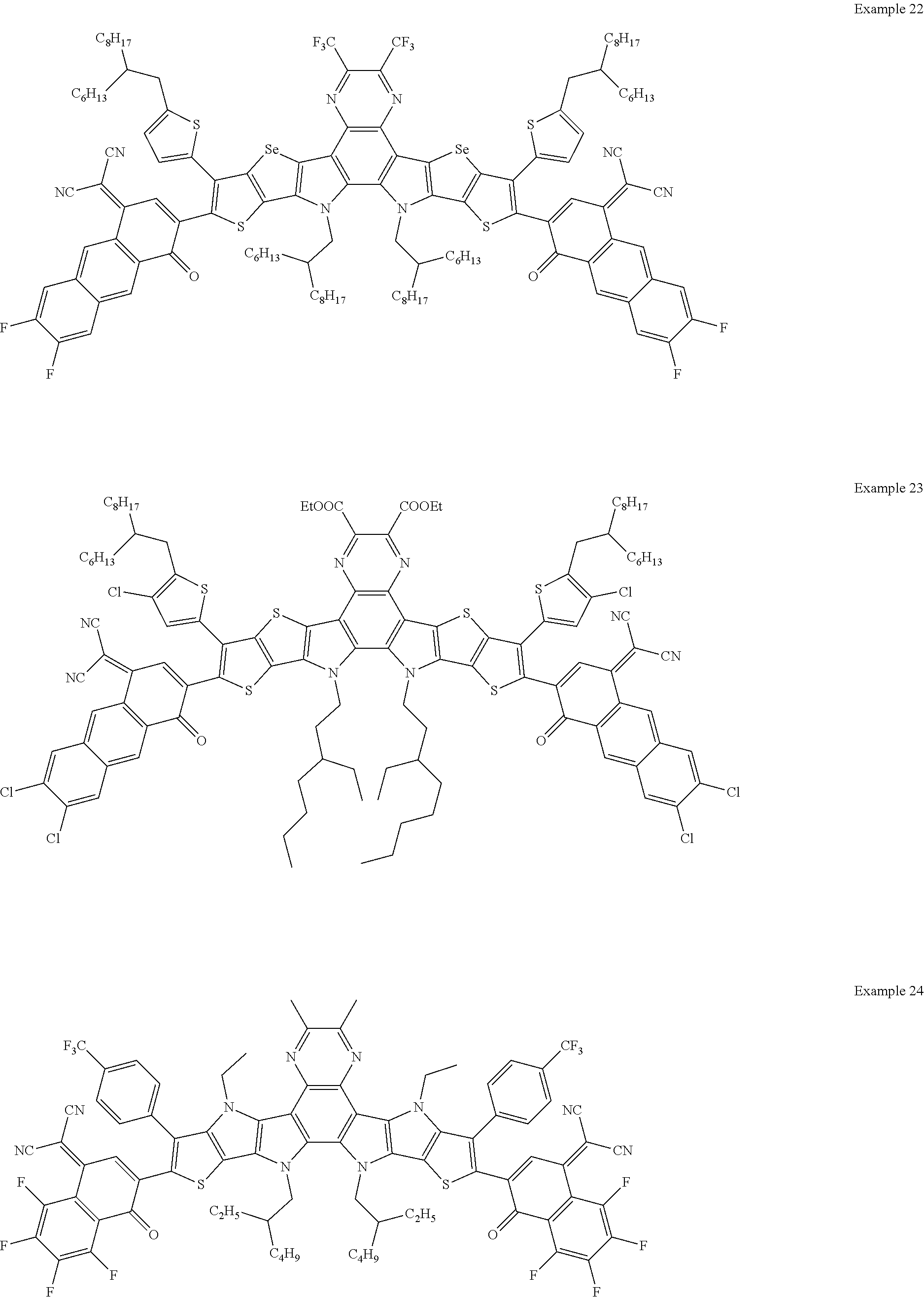

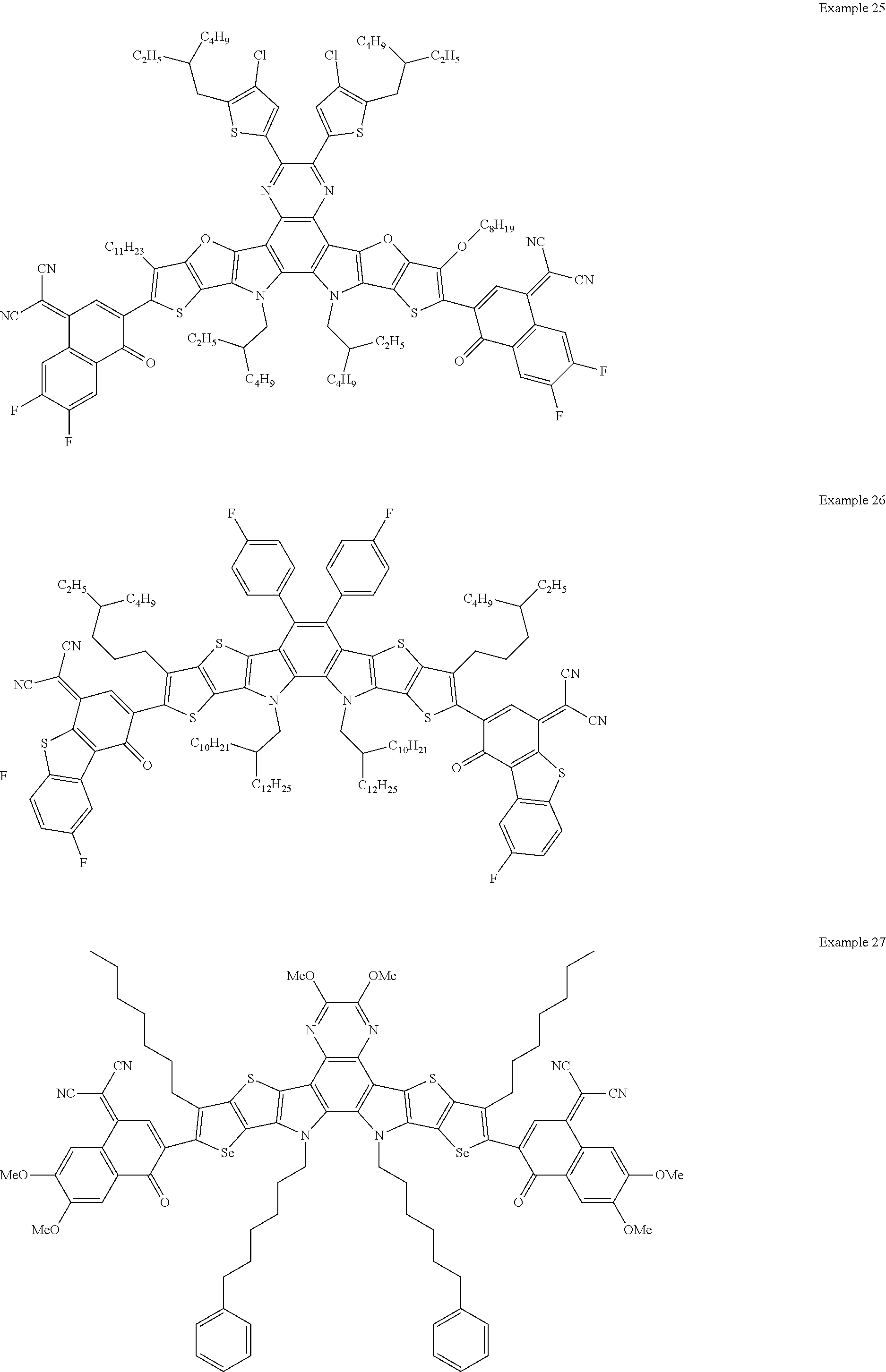

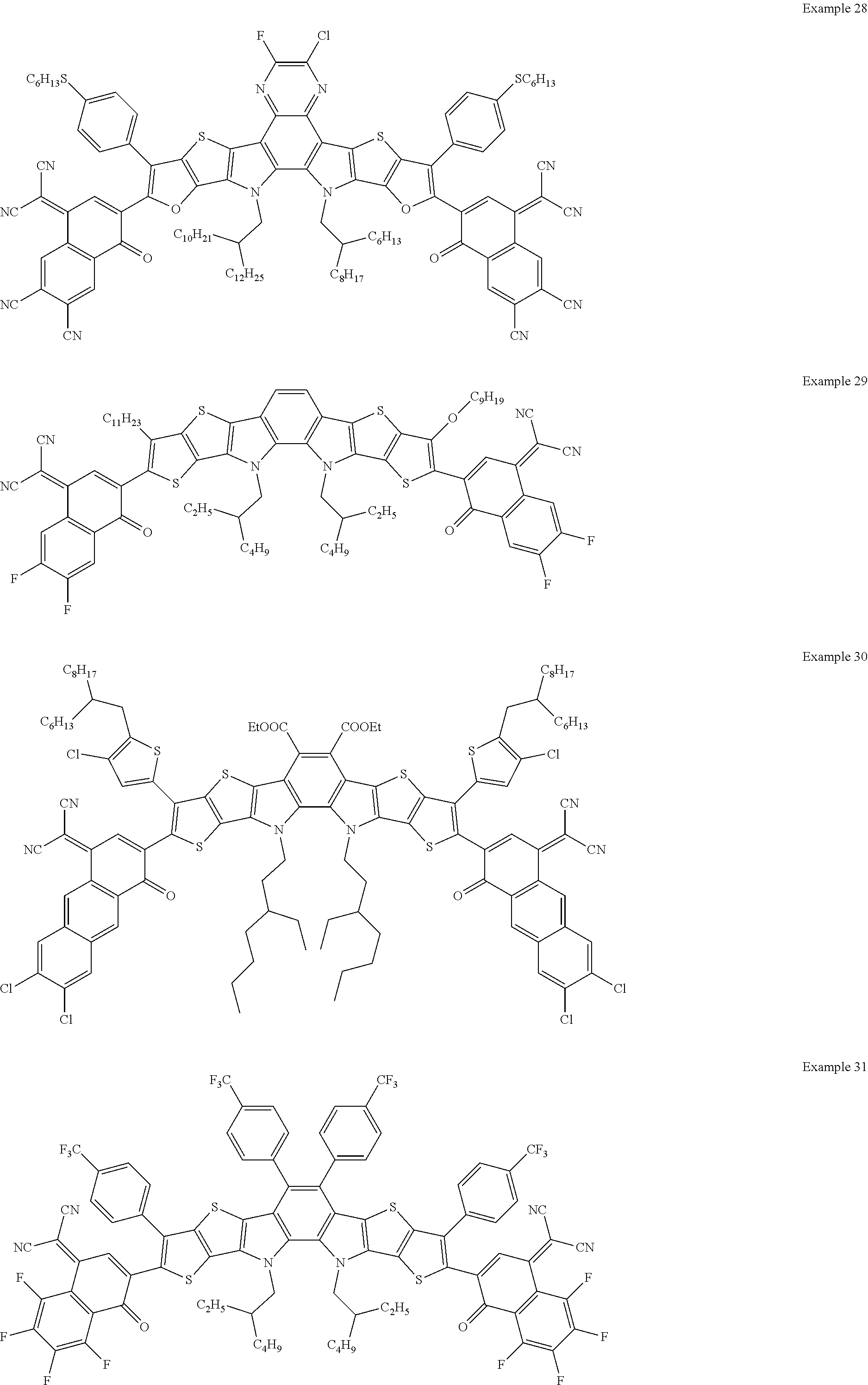

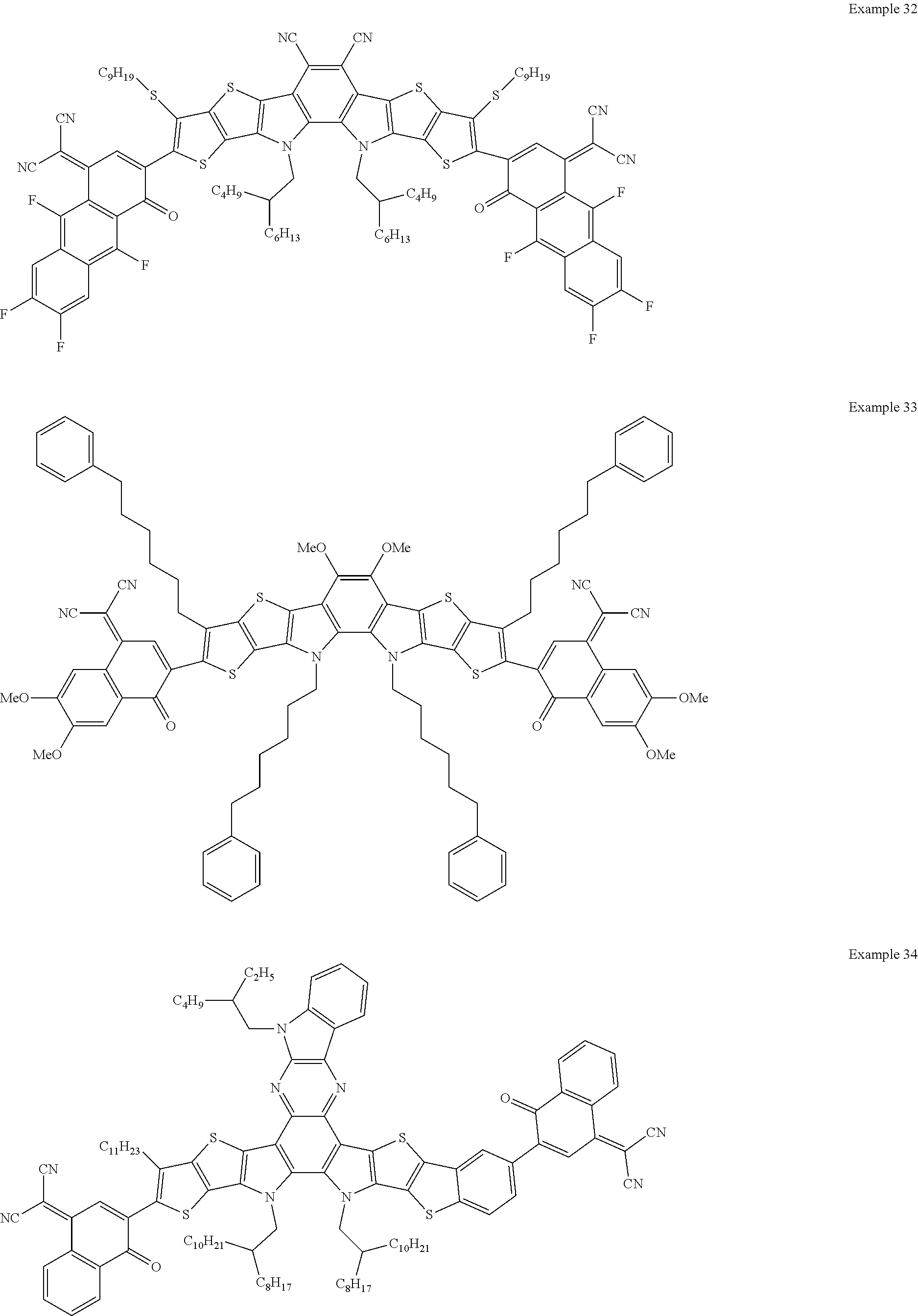

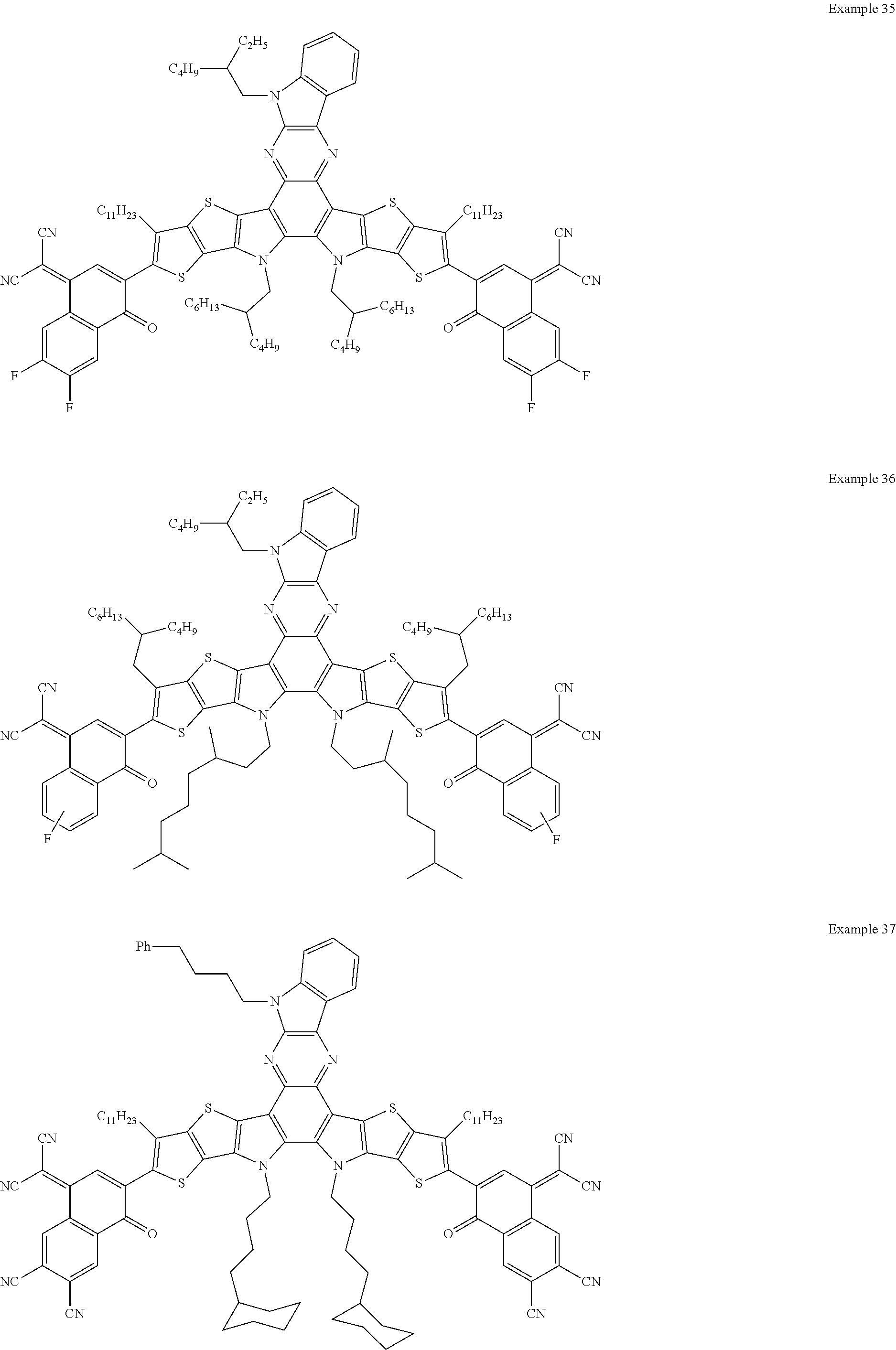

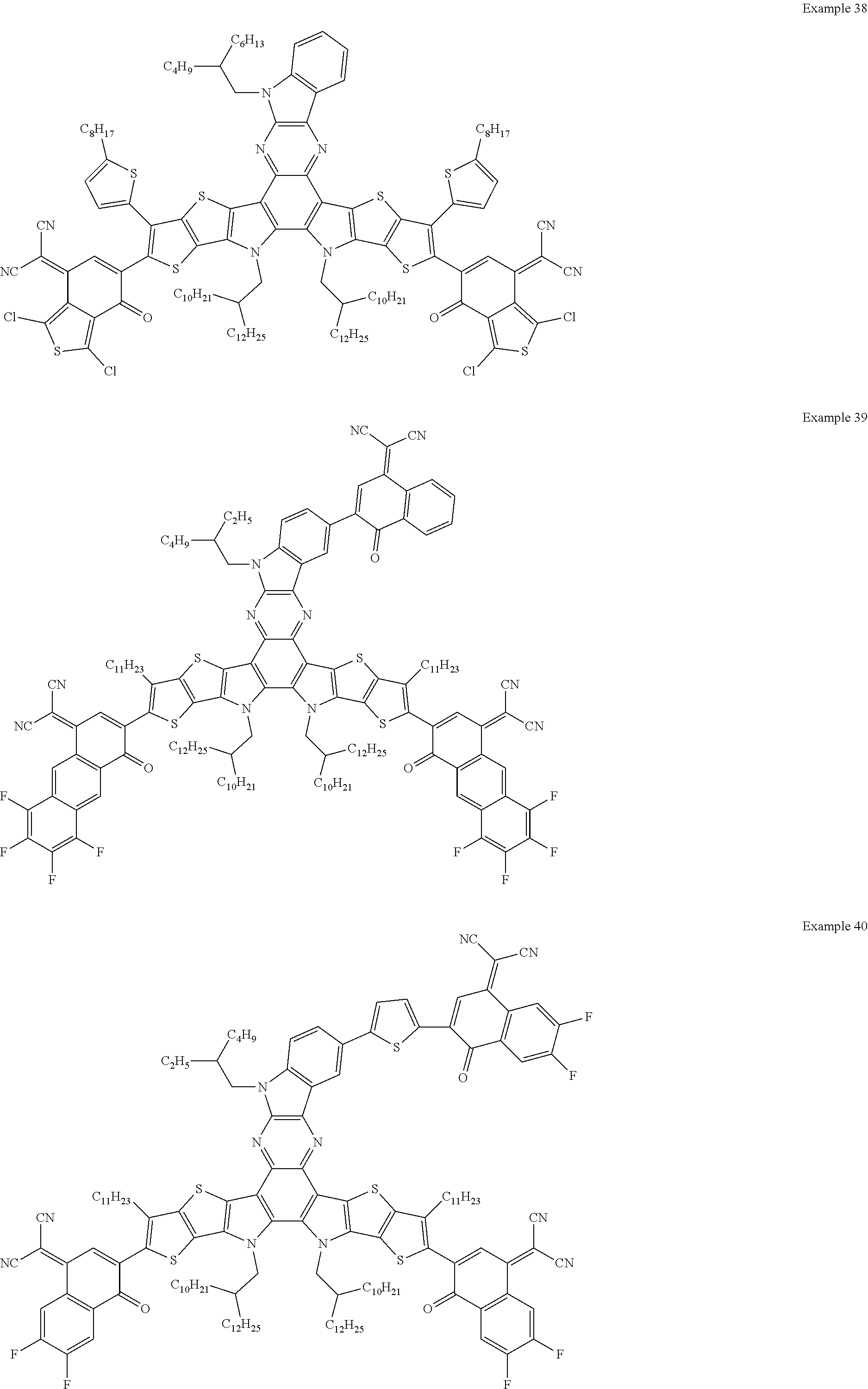

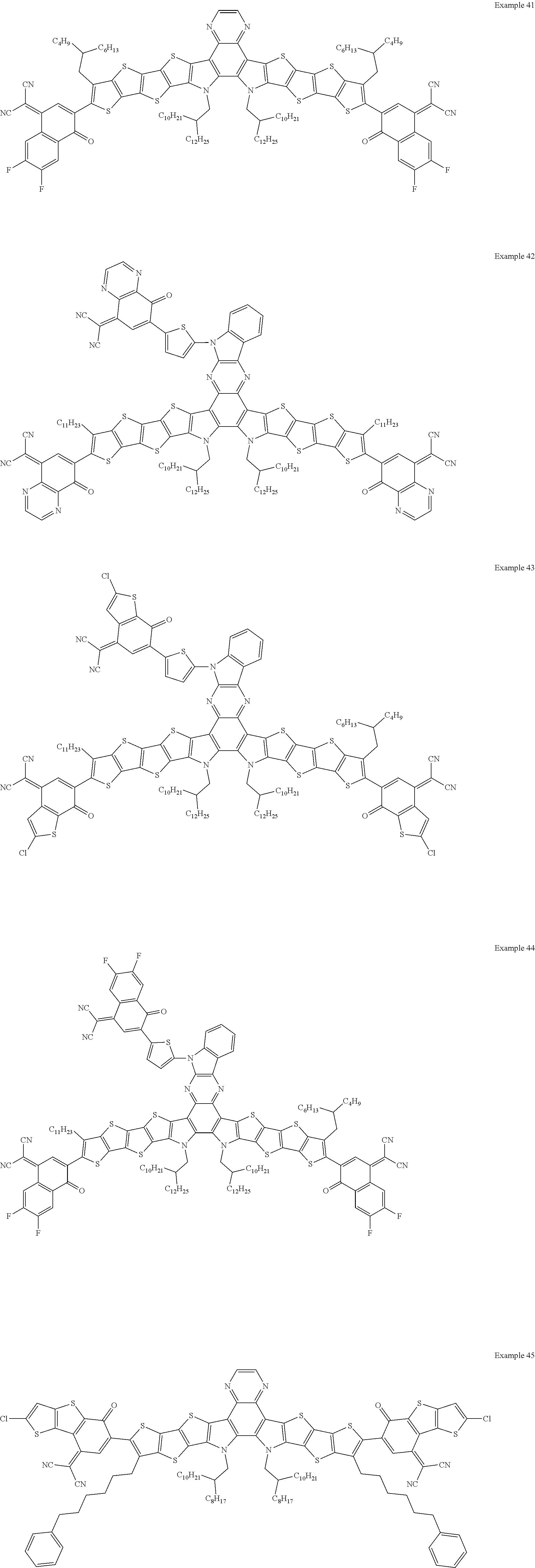

It should be understood that the above-listed embodiments are only intended to allow the person skilled in the art to understand the structure and composition of the present invention more clearly, and are not limited thereto.

In one embodiment, the present invention provides an organic composition comprising at least one P-type organic semiconductor material and at least one N-type organic semiconductor material. The P-type organic semiconductor material comprises at least one organic conjugated polymer or one organic conjugated small molecule. The N-type organic semiconductor material comprises at least one of the aforementioned organic semiconductor materials.

Wherein, the P-type organic semiconductor material is further selected from at least one organic conjugated polymer, and the organic conjugated polymer is formed from a plurality of monomer units, the monomer units being selected from one of the following structures and combinations thereof:

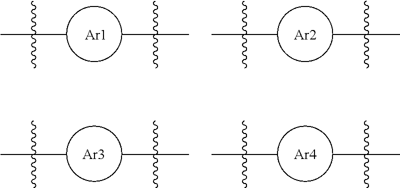

Wherein, Ar1, Ar2, Ar3, and Ar4 are independently selected from a monocyclic ring or a polycyclic ring.

Wherein, the organic conjugated polymer further comprises the following structures and any combinations thereof:

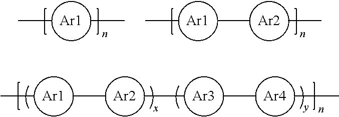

Wherein, Ar1, Ar2, Ar3, and Ar4 are monocyclic rings or polycyclic rings each comprising C3-C30 ring atoms. n is the number of repeating units, and n is a positive integer from 1 to 1000. x and y are mole fractions, wherein 0<x<1, 0<y<1, and x+y=1.

In practice, Ar1 and Ar3 are independently selected from the following structures:

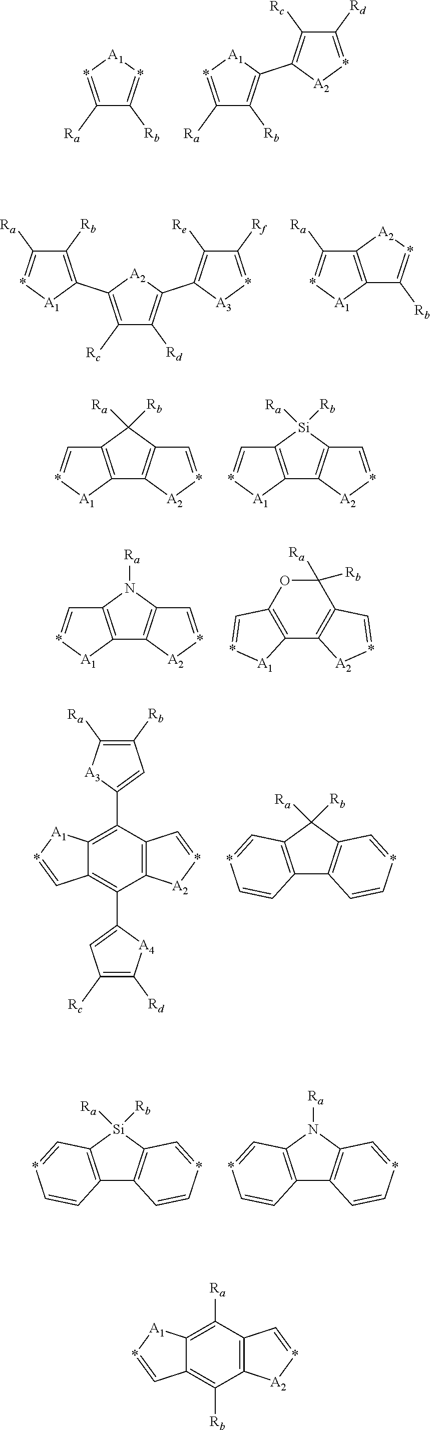

Wherein, A₁, A₂, A₃, and A₄ are independently selected from O, S, and Se. Rₐ, R_{b}, R_{c}, R_{d}, Rₑ, and R_{f} are independently selected from the following groups and any combinations thereof: hydrogen, halogen, cyano, C1-C30 alkyl with or without substituents, C1-C30 alkoxy with or without substituents, C1-C30 alkylthio with or without substituents, C1-C30 haloalkyl with or without substituents, C2-C30 ester, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, and C2-C30 esteryl heteroaryl with or without substituents. * represents a single bond outward bonding.

In a preferred embodiment, Ar1 and Ar3 are independently selected from one of the following structures:

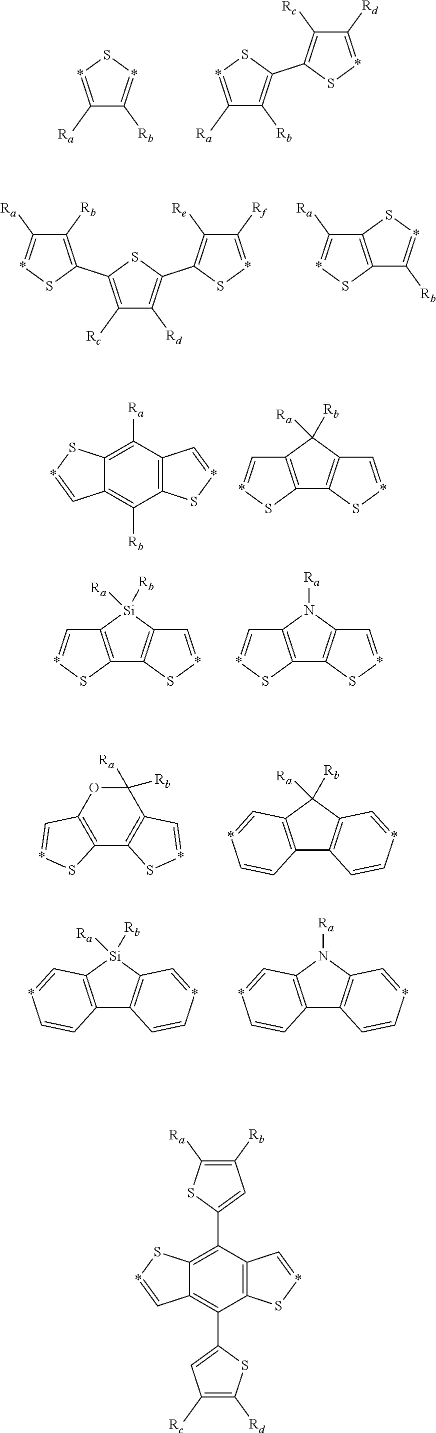

In practice, Ar2 and Ar4 are independently selected from the following structures:

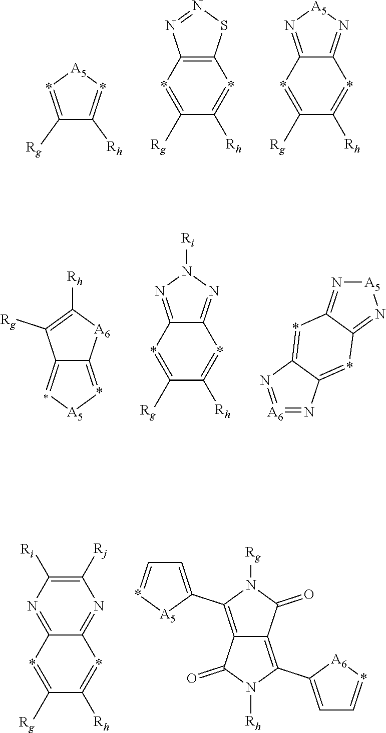

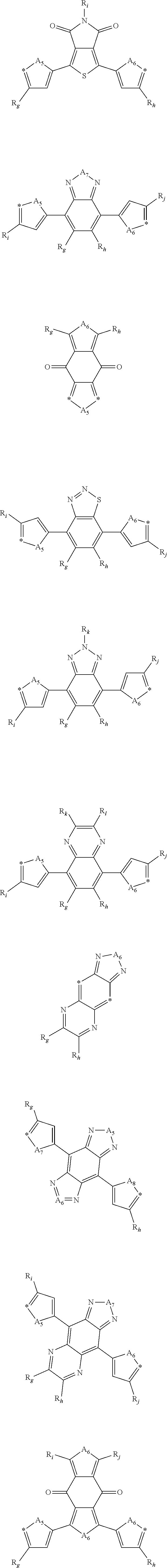

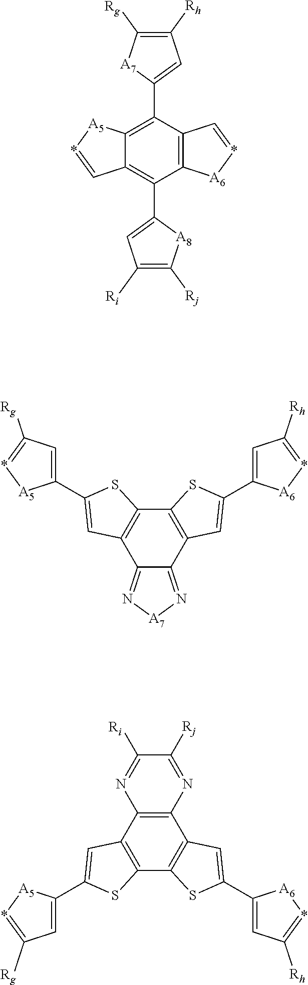

Wherein, A₅, A₆, A₇, and A₈ are independently selected from O, S, and Se. R_{g}, Rₕ, Rᵢ, Rⱼ, Rₖ, and Rₗ are independently selected from the following groups and any combinations thereof: hydrogen, halogen, cyano, C1-C30 alkyl with or without substituents, C1-C30 alkoxy with or without substituents, C1-C30 alkylthio with or without substituents, C1-C30 haloalkyl with or without substituents, C2-C30 ester with or without substituents, C1-C30 alkylaryl with or without substituents, C1-C30 alkyl heteroaryl with or without substituents, C1-C30 silyl aryl with or without substituents, C1-C30 silyl heteroaryl with or without substituents, C1-C30 alkoxyaryl with or without substituents, C1-C30 alkoxy heteroaryl with or without substituents, C1-C30 alkylthioaryl with or without substituents, C1-C30 alkylthio heteroaryl with or without substituents, C1-C30 haloalkyl aryl with or without substituents, C1-C30 haloalkyl heteroaryl with or without substituents, C2-C30 esteryl aryl with or without substituents, and C2-C30 esteryl heteroaryl with or without substituents. * represents a single bond outward bonding.

In a preferred embodiment, Ar2 and Ar4 are independently selected from one of the following structures:

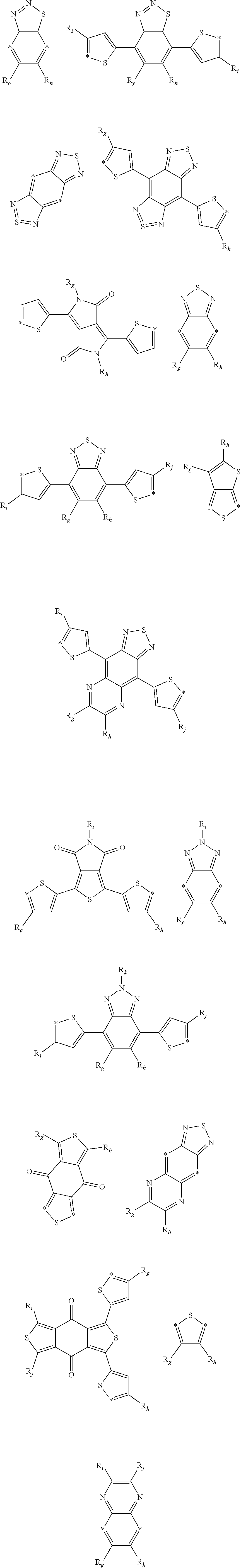

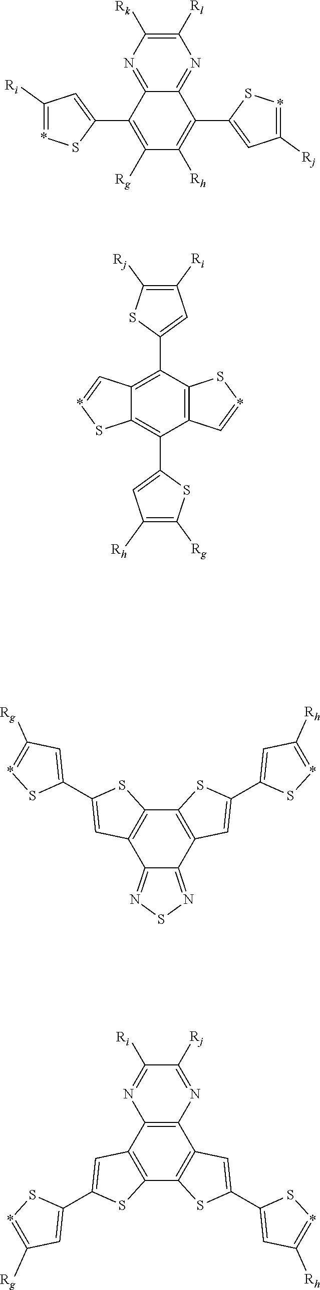

The substituents mentioned above can be independently selected from the following groups and their derivatives: C1-C30 alkyl, C3-C30 branched alkyl, C1-C30 silyl, C2-C30 ester, C1-C30 alkoxy, C1-C30 alkylthio, C1-C30 haloalkyl, C2-C30 olefin, C2-C30 alkyne, C2-C30 carbon chains containing cyano group, C1-C30 carbon chains containing nitro groups, C1-C30 carbon chains containing hydroxy groups, C3-C30 carbon chains containing keto groups, halogens, cyano groups, hydrogen, deuterium and tritium. The above-mentioned aryl group and heteroaryl group may have a monocyclic or polycyclic structure.

In practice, the conjugated polymer further comprises the following embodiments D1˜D39:

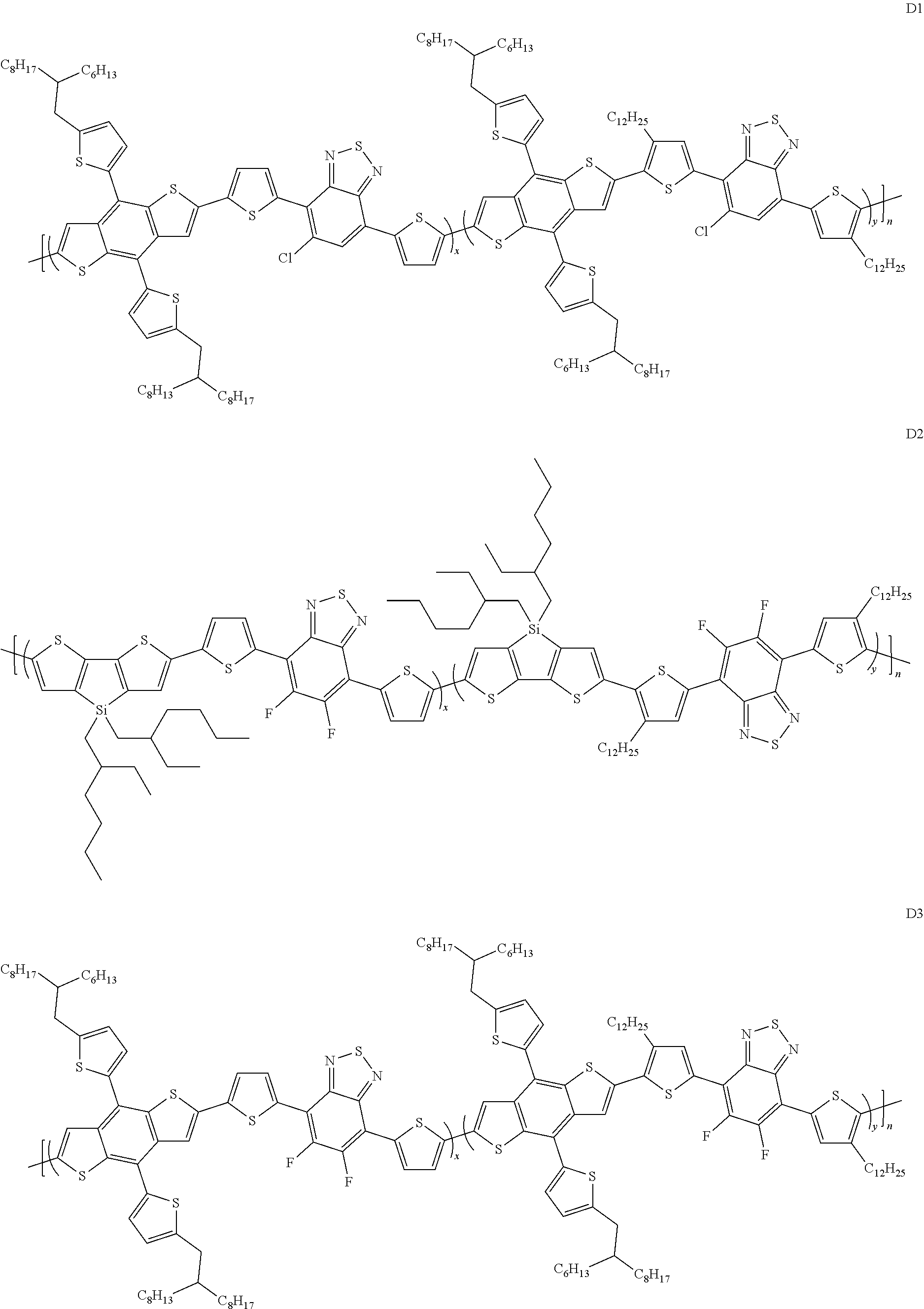

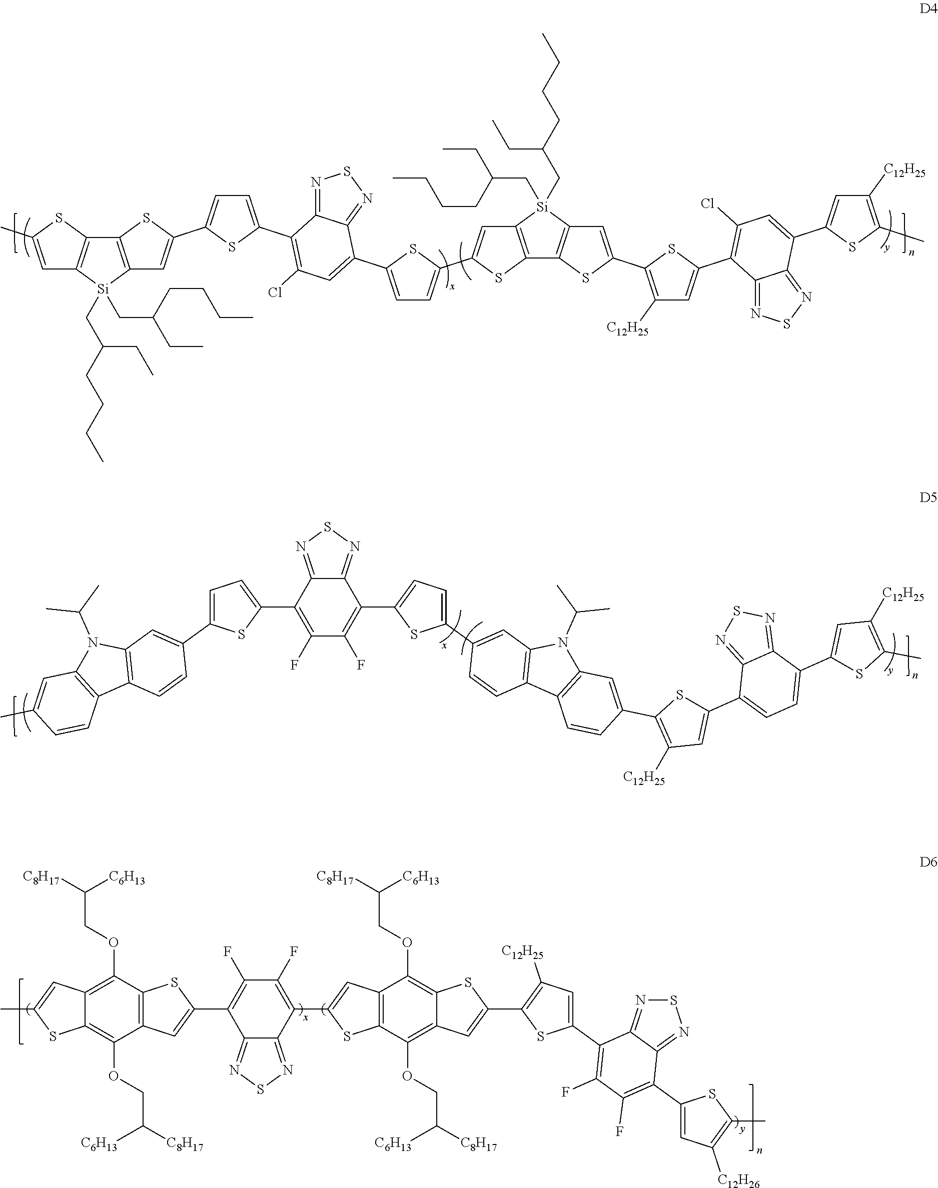

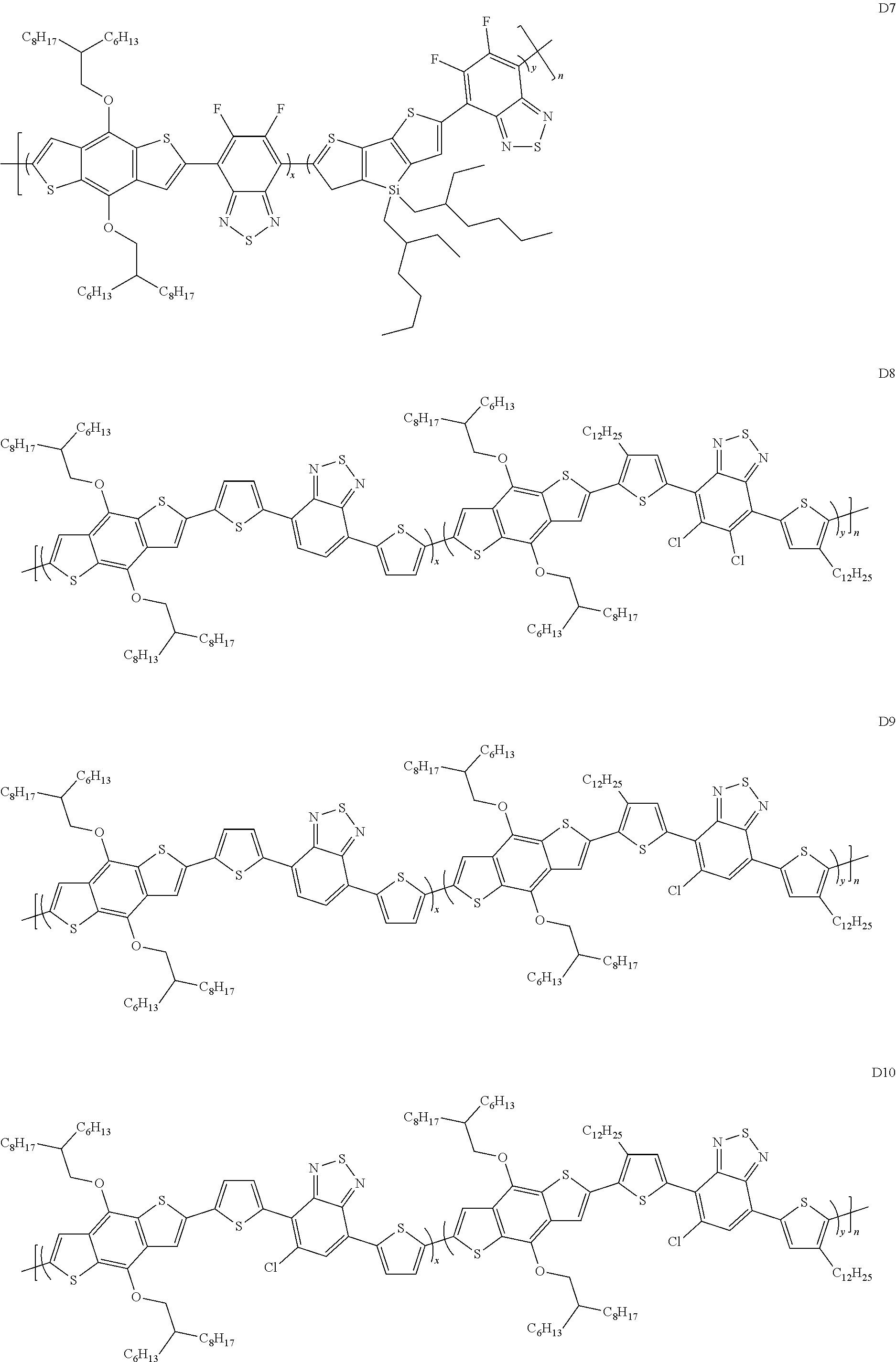

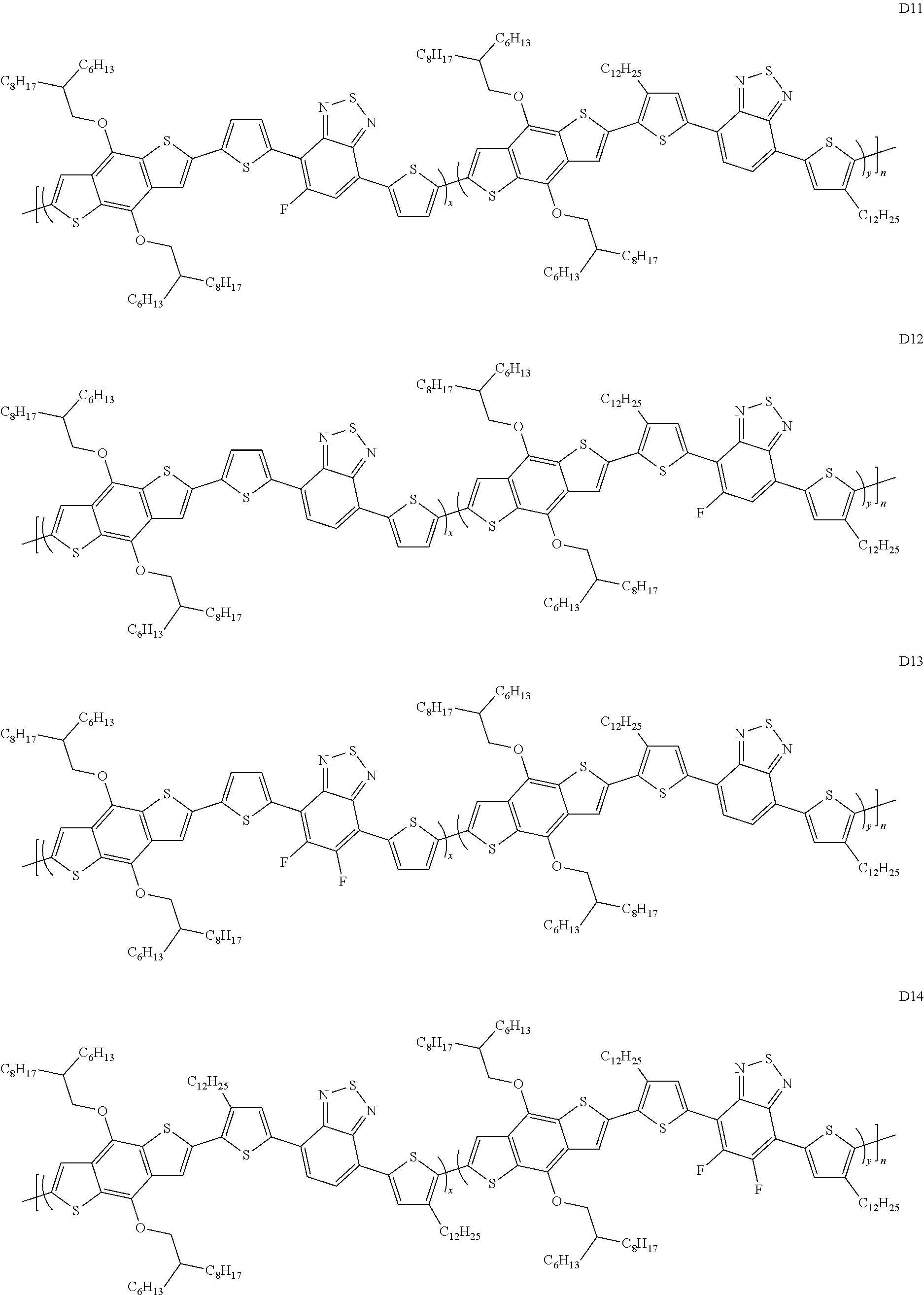

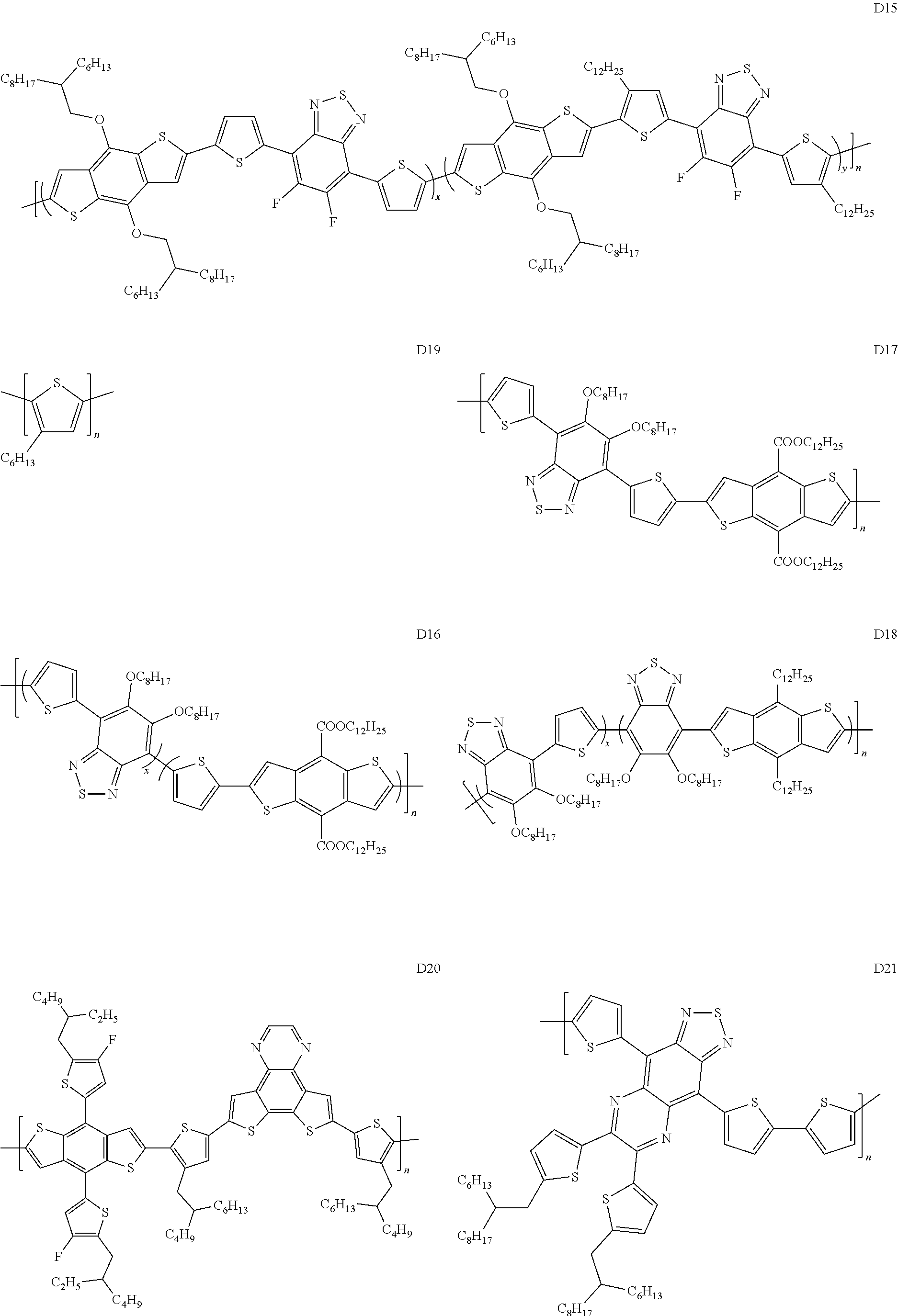

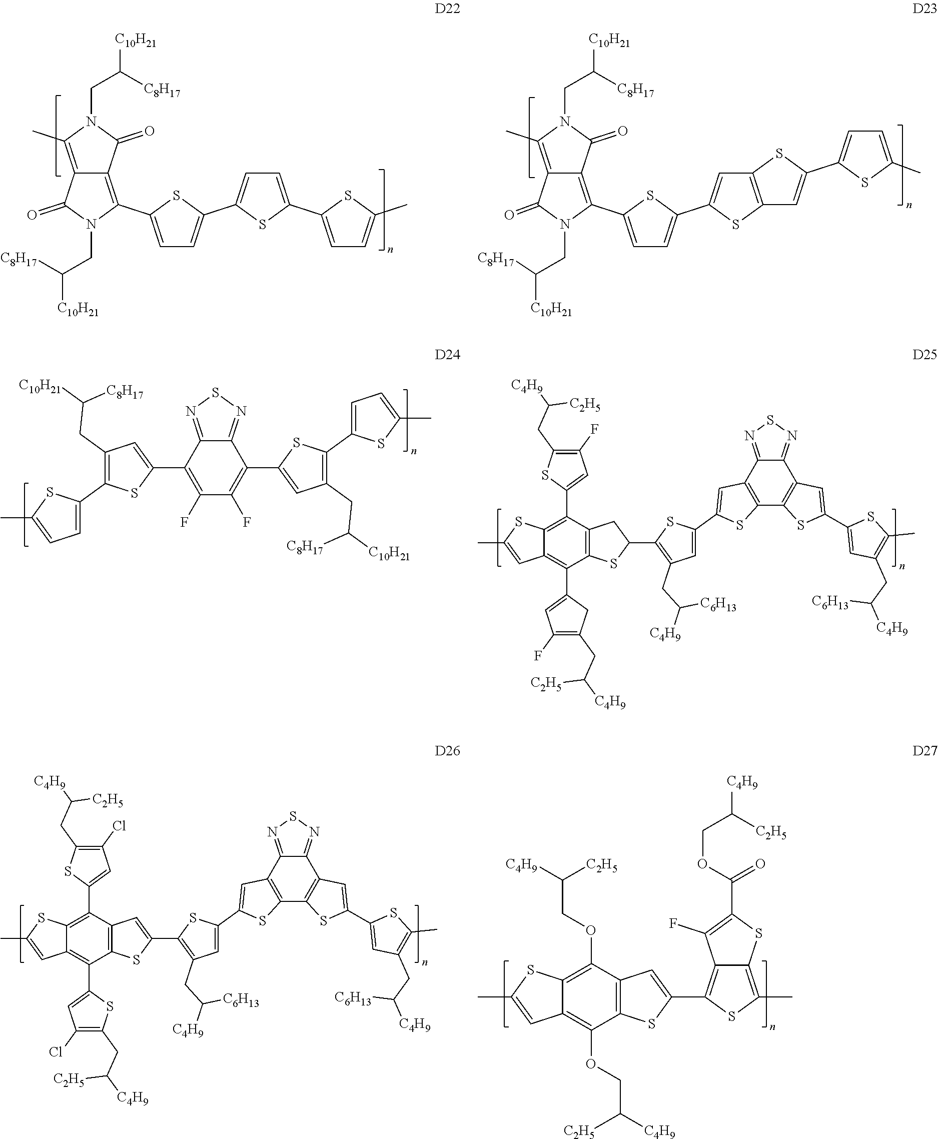

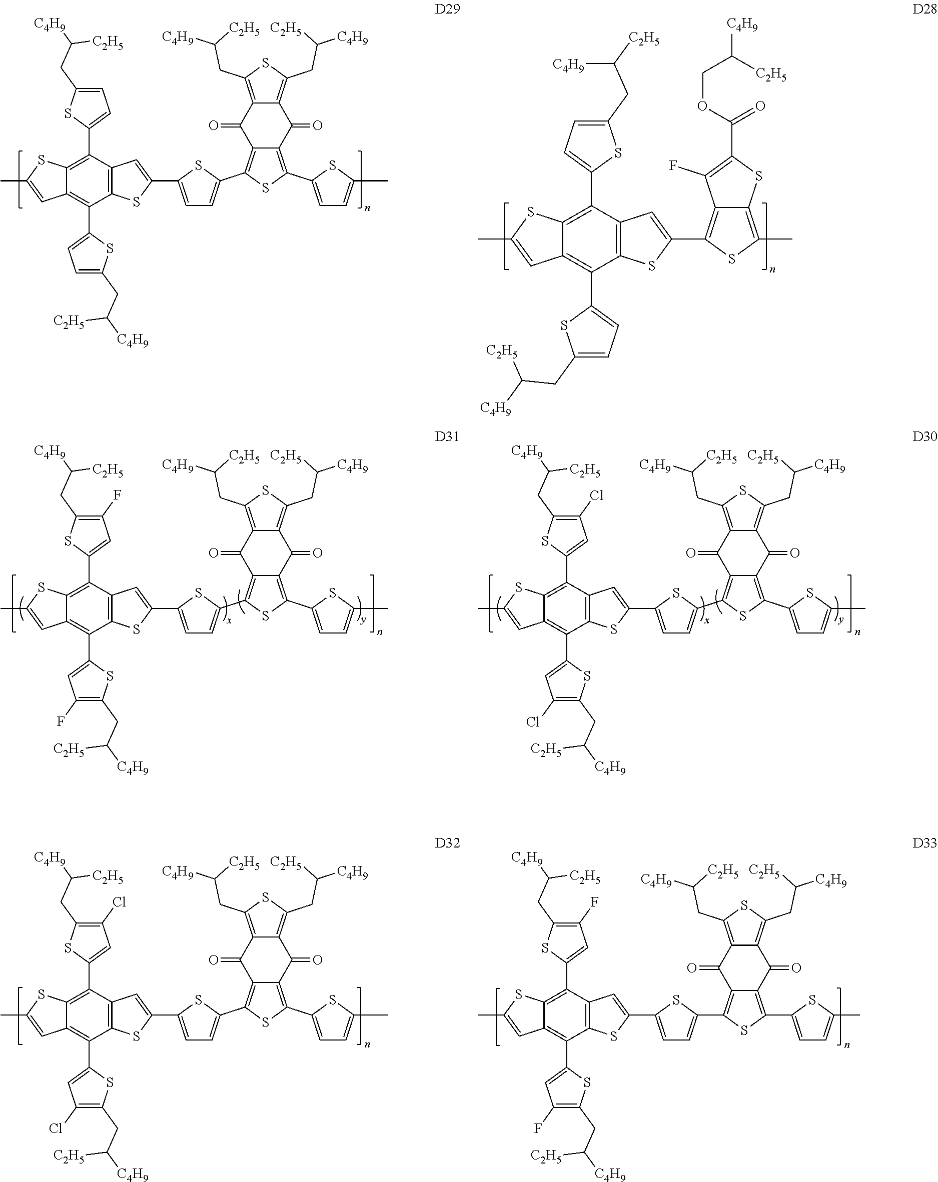

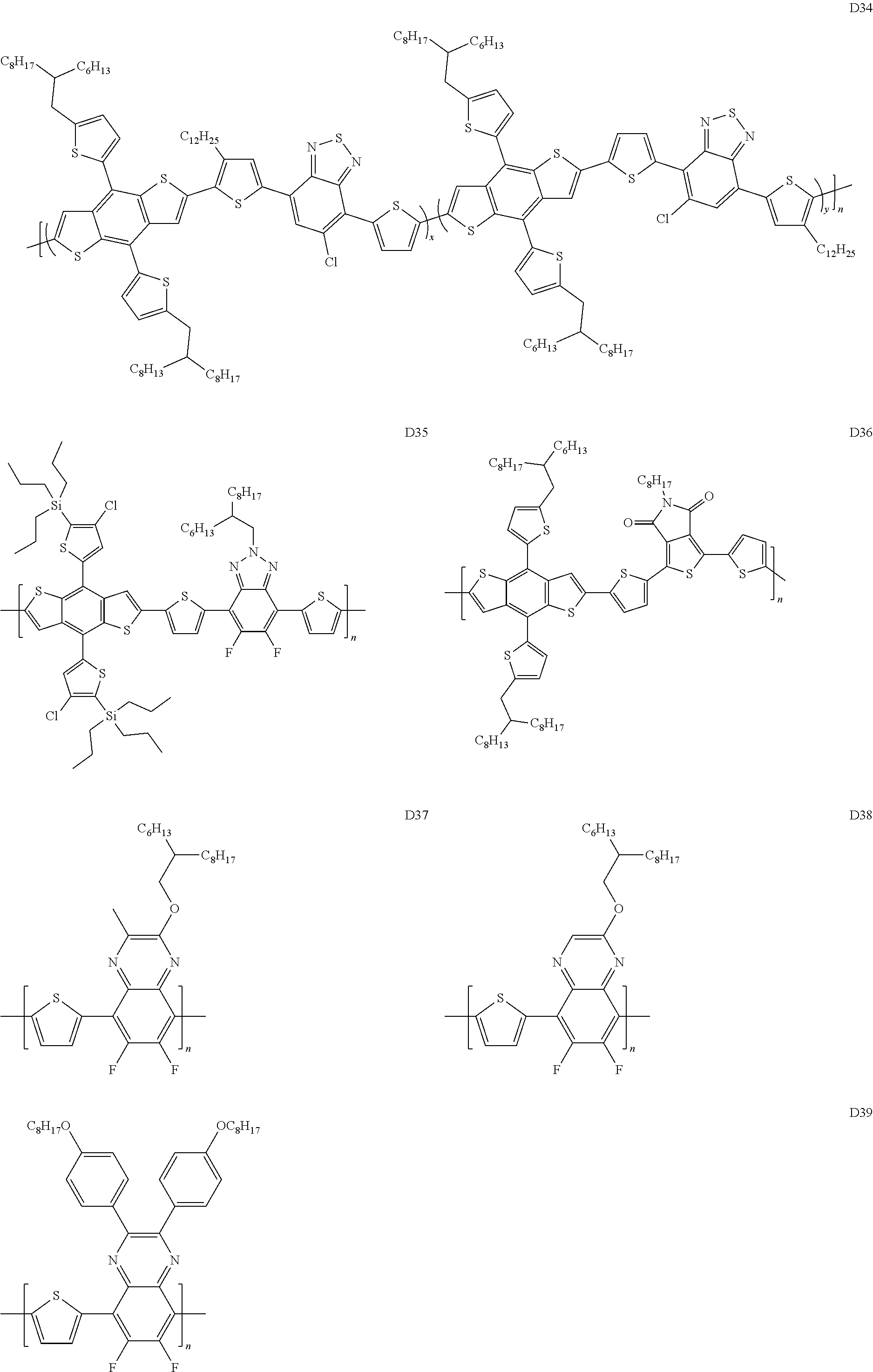

It should be understood that the above-listed embodiments are only intended to allow the person skilled in the art to understand the structure and composition of the present invention more clearly, and are not limited thereto.

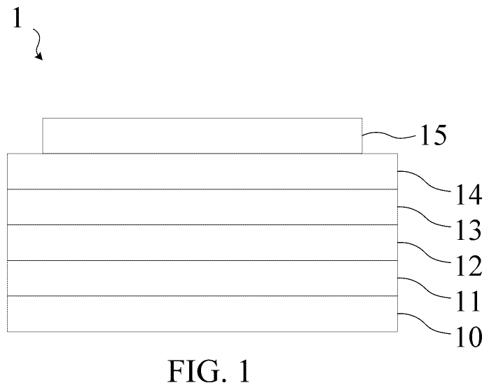

Please refer to FIG. 1. FIG. 1 shows a schematic structural diagram of one embodiment of an organic optoelectronic device of the present invention. As shown in FIG. 1, in another embodiment, the present invention further provides an organic optoelectronic device 1, which comprises a first electrode 11, a second electrode 15 and an active layer 13. The active layer 13, which comprises at least one of the organic semiconductor material previously and the organic composition previously comprising Formula I, is disposed between the first electrode 11 and the second electrode 15. The organic optoelectronic device 1 further comprises a first carrier transporting layer 12 and a second carrier transporting layer 14. The organic optoelectronic device 1 may have a stacked structure, which sequentially includes a substrate 10, the first electrode 11 (transparent or semi-transparent electrode), the first carrier transporting layer 12, the active layer 13, the second carrier transporting layer 14 and the second electrode 15. The first carrier transporting layer 12 is configured to transport carriers in the first electrode 11 and the active layer 13, and the second carrier transporting layer 14 is configured to transport carriers in the active layer 13 and the second electrode 15. Specifically, the first carrier transporting layer 12 is one of an electron transporting layer and a hole transporting layer, and the second carrier transporting layer 14 is the other one. In detail, when the first carrier transporting layer 12 is the electron transporting layer, the second carrier transporting layer 14 is the hole transporting layer, which is an inverted stacked structure; when the first carrier transporting layer 12 is the hole transporting layer, the second carrier transporting layer 14 is an electron transporting layer, which is a conventional stacked structure. In practice, the organic optoelectronic device 1 may comprise an organic photovoltaic device, an organic photodetector device, or an organic light emitting diode.

In order to illustrate the organic composition of the present invention more clearly, the following experiments will be conducted using Comparative Example Y-QC4F and organic semiconductor materials Example 1˜Example 9 of the invention to illustrate the differences in efficacy. These materials will then be further utilized as an N-type organic semiconductor material combined with a P-type organic semiconductor material to prepare the organic composition. The active layers comprising the organic semiconductor materials or organic compositions previously described will be fabricated into organic optoelectronic devices for material testing and device performance evaluation.

For the optical physical quality testing part of material testing and device testing, the UV absorption spectrum measurement instrument model is Hitachi UH5700, and the oxidation potential is measured by using cyclic voltammetry with CH Instrument 611E.

### Synthesis of Example 1

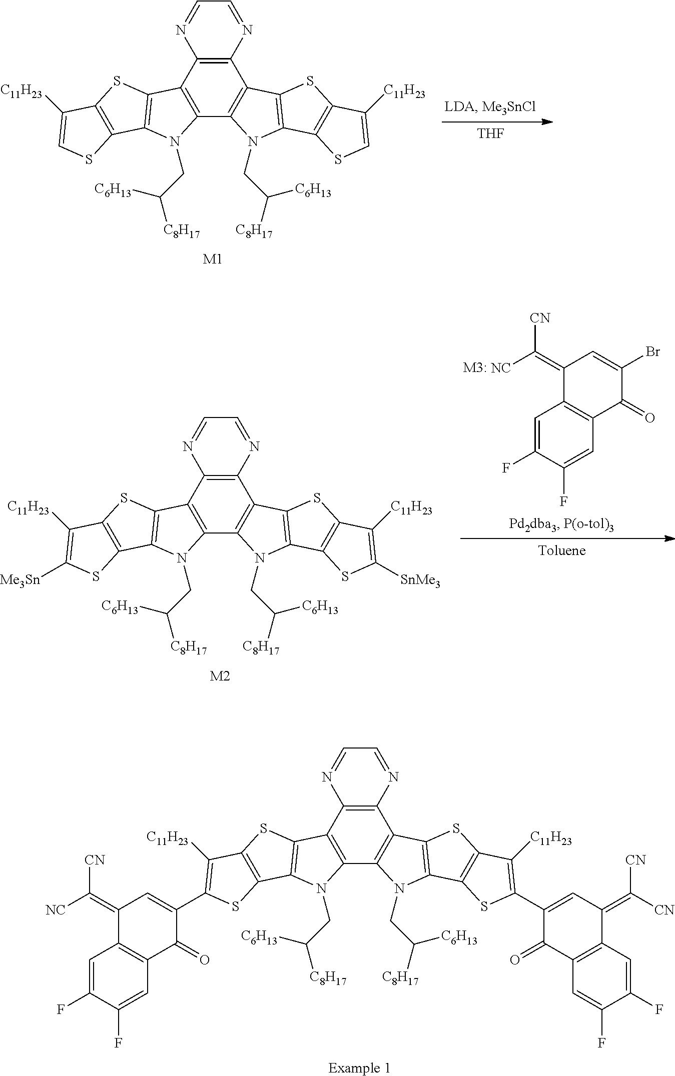

M1 (560 mg, 0.47 mmol) and anhydrous tetrahydrofuran (THF) were added into a dried 100 mL three-neck flask. The mixture was cooled to 0° C., followed by the addition of 2.0 M lithium diisopropylamide (LDA, 1.87 mL, 3.76 mmol). The reaction was maintained below 0° C. for 30 minutes. Trimethyltin chloride (Me₃SnCl, 840 mg, 4.24 mmol, completely dissolved in anhydrous THF) was added at 0° C. After stirring at room temperature for 16 hours, water was added to quench the reaction. The mixture was extracted with heptane/water three times. The organic layers were dried over magnesium sulfate, and the solvent was removed to afford the product M2 as an orange oil (710 mg, yield: 99%). ¹H NMR (600 MHz, CDCl₃): δ 8.91 (s, 2H), 4.66 (d, J=7.8 Hz, 4H), 2.81 (t, J=8.1 Hz, 4H), 2.14-2.12 (m, 2H), 1.86-1.83 (m, 4H), 1.52-0.74 (m, 92H), 0.67 (t, J=7.2 Hz, 6H), 0.47 (s, 18H). M2 (710 mg, 0.47 mmol) and M3 (376 mg, 1.17 mmol) were added to a 100 mL two-neck flask, followed by the addition of toluene (30 mL). The mixture was degassed at room temperature for 30 minutes. Tris(dibenzylideneacetone)dipalladium (Pd₂(dba)₃, 17.2 mg, 0.019 mmol) and tris(o-tolyl)phosphine (P(o-tol)₃, 22.8 mg, 0.075 mmol) were added. The reaction mixture was heated to 90° C. and stirred for 18 hours, then cooled to room temperature. The crude mixture was purified by silica gel column chromatography (eluent: heptane/dichloromethane=2/1) to afford Example 1 as a black solid (196 mg, yield: 25%). ¹H NMR (500 MHz, CDCl₃): δ 8.98 (s, 2H), 8.78-8.74 (m, 2H), 8.20-8.17 (m, 2H), 8.05 (s, 2H), 4.72 (d, J=8.0 Hz, 4H), 3.12 (t, J=8.3 Hz, 4H), 2.17-2.13 (m, 2H), 2.02-1.98 (m, 4H), 1.60-1.55 (m, 4H), 1.33-0.76 (m, 88H), 0.69-0.66 (m, 6H).

### Synthesis of Example 2

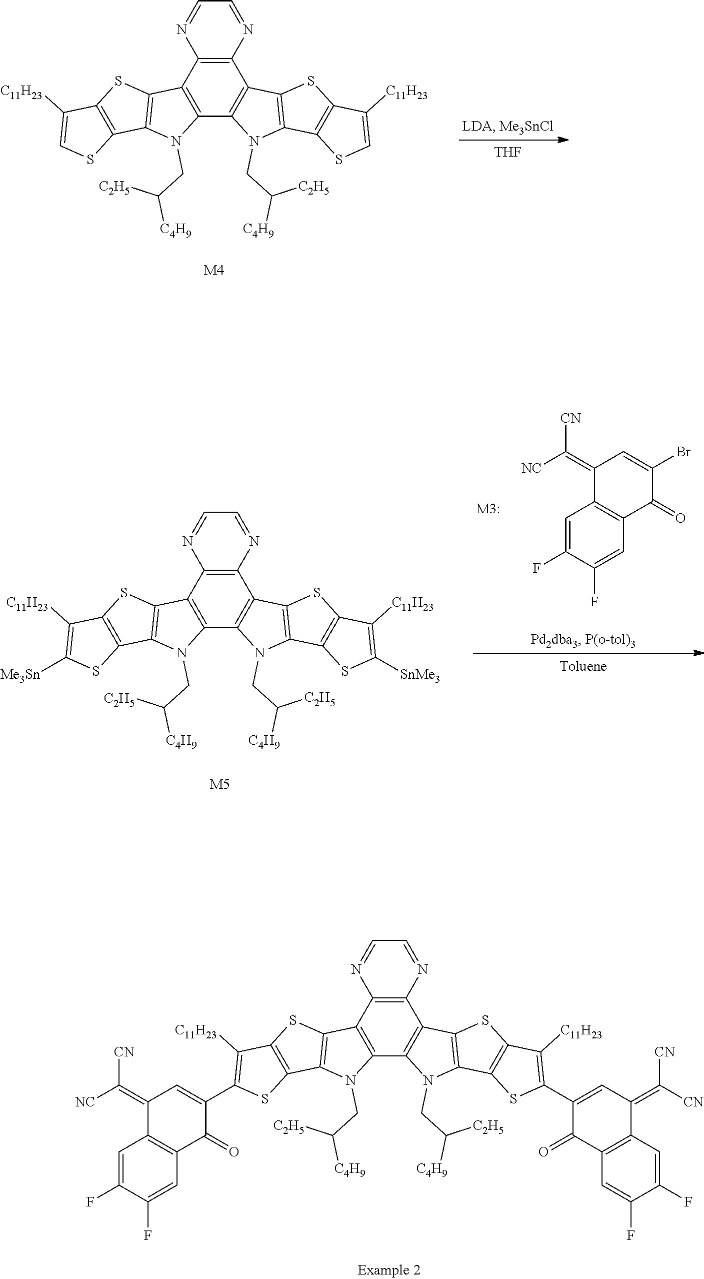

M4 (2.1 g, 2.17 mmol) and anhydrous THF were added into a dried 100 mL three-neck flask. The mixture was cooled to 0° C., followed by the addition of 2.0 M LDA (8.7 mL, 17.4 mmol). The reaction was maintained below 0° C. for 30 minutes. Me₃SnCl (3.9 g, 19.6 mmol, completely dissolved in anhydrous THF) was added at 0° C. After stirring at room temperature for 16 hours, water was slowly added to quench the reaction. The mixture was extracted with heptane/water three times. The organic layers were dried over magnesium sulfate, and the solvent was removed to afford M5 as an orange oil (2.8 g, yield: 99%). ¹H NMR (500 MHz, CDCl₃): δ 8.92 (s, 2H), 4.69-4.65 (m, 4H), 2.82 (t, J=8.0 Hz, 4H), 2.11-2.05 (m, 2H), 1.87-1.81 (m, 4H), 1.44-0.86 (m, 54H), 0.63-0.61 (m, 12H), 0.48 (s, 18H). M5 (980 mg, 0.76 mmol) and M3 (609 mg, 1.90 mmol) were added into a 100 mL two-neck flask, followed by the addition of toluene (50 mL). The mixture was degassed at room temperature for 30 minutes. Pd₂(dba)₃ (69.5 mg, 0.076 mmol) and P(o-tol)₃ (92.4 mg, 0.304 mmol) were added. The reaction mixture was heated to 90° C. and stirred for 18 hours, then cooled to room temperature. Purification by silica gel column chromatography (eluent: heptane/dichloromethane=2/1) afforded Example 2 as a black solid (100 mg, yield: 9%). ¹H NMR (500 MHz, CDCl₃): δ 8.98 (s, 2H), 8.77-8.74 (m, 2H), 8.21-8.17 (m, 2H), 8.05 (s, 2H), 4.77-4.68 (m, 4H), 3.12 (t, J=8.3 Hz, 4H), 2.13-2.10 (m, 2H), 2.04-1.99 (m, 4H), 1.41-0.71 (m, 54H), 0.66-0.64 (m, 12H).

### Synthesis of Example 3

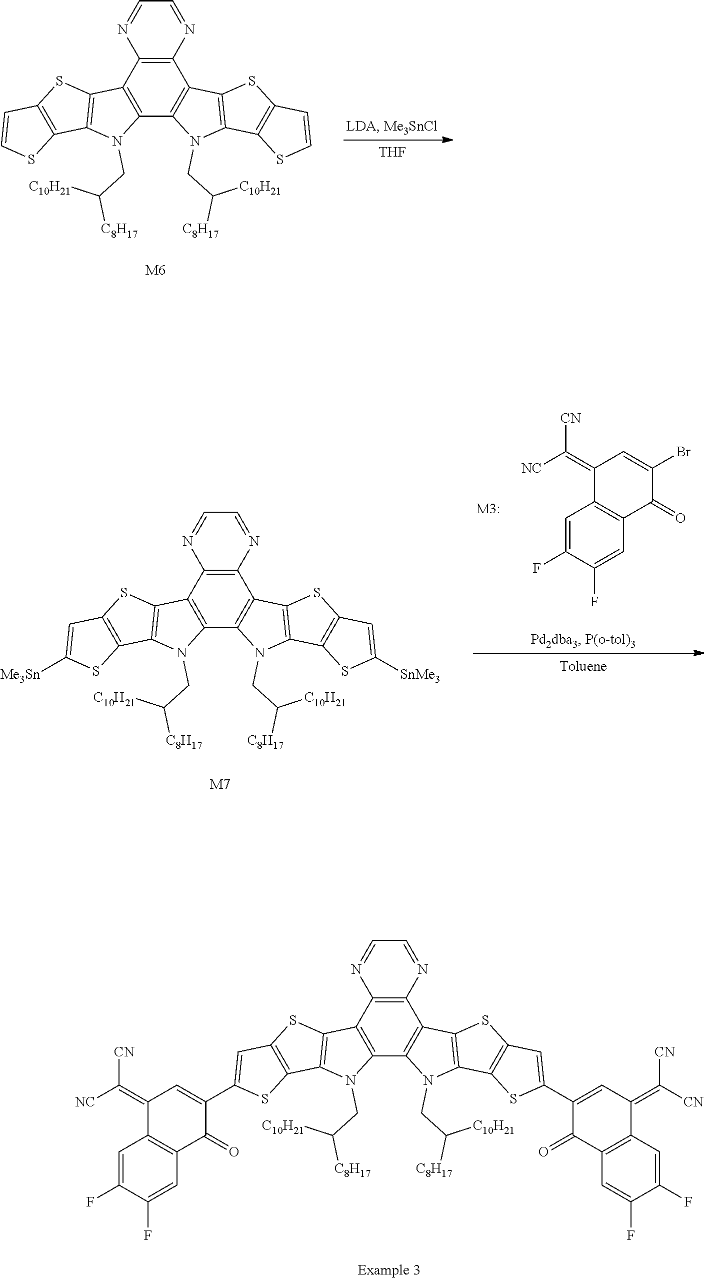

M6 (350 mg, 0.35 mmol) and anhydrous THF were added into a dried 100 mL three-neck flask. The mixture was cooled to 0° C., followed by the addition of 2.0 M LDA (1.40 mL, 2.82 mmol). The reaction was maintained below 0° C. for 30 minutes. Me₃SnCl (632 mg, 3.17 mmol, completely dissolved in anhydrous THF) was added at 0° C. After stirring at room temperature for 16 hours, water was slowly added to quench the reaction. The mixture was extracted with heptane/water three times. The organic layers were dried over magnesium sulfate, and the solvent was removed to afford M7 as an orange oil (500 mg, yield: 99%). ¹H NMR (600 MHz, CDCl₃): δ 8.90 (s, 2H), 7.47 (s, 2H), 4.68 (d, J=7.8 Hz, 4H), 2.15-2.09 (m, 2H), 1.30-0.65 (m, 76H), 0.47 (s, 18H). M7 (500 mg, 0.38 mmol) and M3 (304 mg, 0.95 mmol) were added to a 100 mL two-neck flask, followed by the addition of toluene (30 mL). The mixture was degassed at room temperature for 30 minutes. Pd₂(dba)₃ (13.9 mg, 0.015 mmol) and P(o-tol)₃ (18.5 mg, 0.061 mmol) were added. The reaction mixture was heated to 90° C. and stirred for 18 hours, then cooled to room temperature. Purification by silica gel column chromatography (eluent: heptane/dichloromethane=2/1) afforded Example 3 as a black solid (150 mg, yield: 27%). ¹H NMR (500 MHz, CDCl₃): δ 8.97 (s, 2H), 8.81-8.77 (m, 2H), 8.38 (s, 2H), 8.22-8.18 (m, 4H), 4.73 (d, J=7.5 Hz, 4H), 2.18-2.14 (m, 2H), 1.28-0.77 (m, 76H).

### Synthesis of Example 4

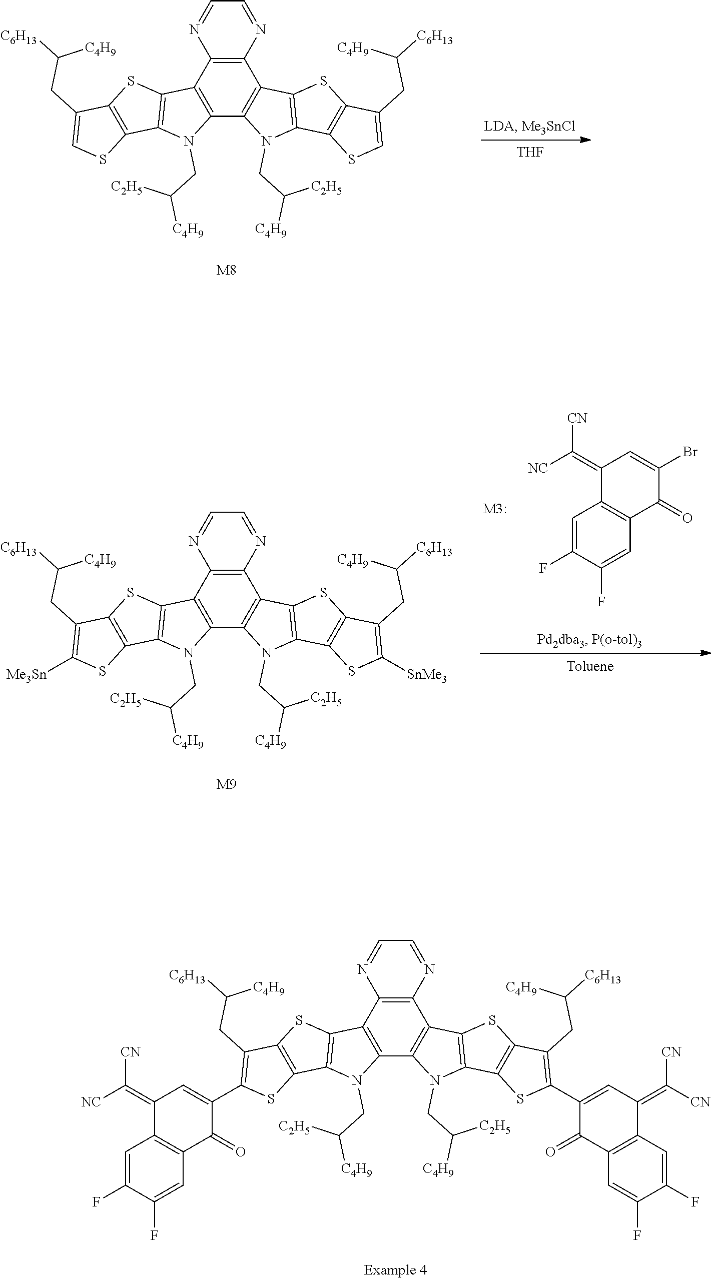

M8 (800 mg, 0.81 mmol) and anhydrous THF were added into a dried 100 mL three-neck flask. The mixture was cooled to 0° C., followed by the addition of 2.0 M LDA (3.22 mL, 6.44 mmol). The reaction was maintained below 0° C. for 30 minutes. Me₃SnCl (1.44 g, 7.25 mmol, completely dissolved in anhydrous THF) was added at 0° C. After stirring at room temperature for 16 hours, water was slowly added to quench the reaction. The mixture was extracted with heptane/water three times. The organic layers were dried over magnesium sulfate, and the solvent was removed to afford M9 as an orange oil (1.1 g, yield: 99%). ¹H NMR (600 MHz, CDCl₃): δ 8.92 (s, 2H), 4.68-4.64 (m, 4H), 2.76 (d, J=7.2 Hz, 4H), 2.15-2.09 (m, 4H), 1.36-0.84 (m, 60H), 0.66-0.60 (m, 12H), 0.48 (s, 18H). M9 (550 mg, 0.42 mmol) and M3 (335 mg, 1.04 mmol) were added into a 100 mL two-neck flask, followed by the addition of toluene (30 mL). The mixture was degassed at room temperature for 30 minutes. Pd₂(dba)₃ (38.2 mg, 0.042 mmol) and P(o-tol)₃ (50.8 mg, 0.167 mmol) were added. The reaction mixture was heated to 90° C. and stirred for 18 hours, then cooled to room temperature. Purification by silica gel column chromatography (eluent: heptane/dichloromethane=2/1) afforded Example 4 as a black solid (60 mg, yield: 10%). ¹H NMR (600 MHz, CDCl₃): δ 8.97 (s, 2H), 8.77-8.74 (m, 2H), 8.21-8.18 (m, 2H), 8.15 (s, 2H), 4.75-4.68 (m, 4H), 3.12 (d, J=7.8 Hz, 4H), 2.13-2.10 (m, 4H), 1.32-0.79 (m, 60H), 0.74-0.70 (m, 6H), 0.65-0.62 (m, 6H).

### Synthesis of Example 5

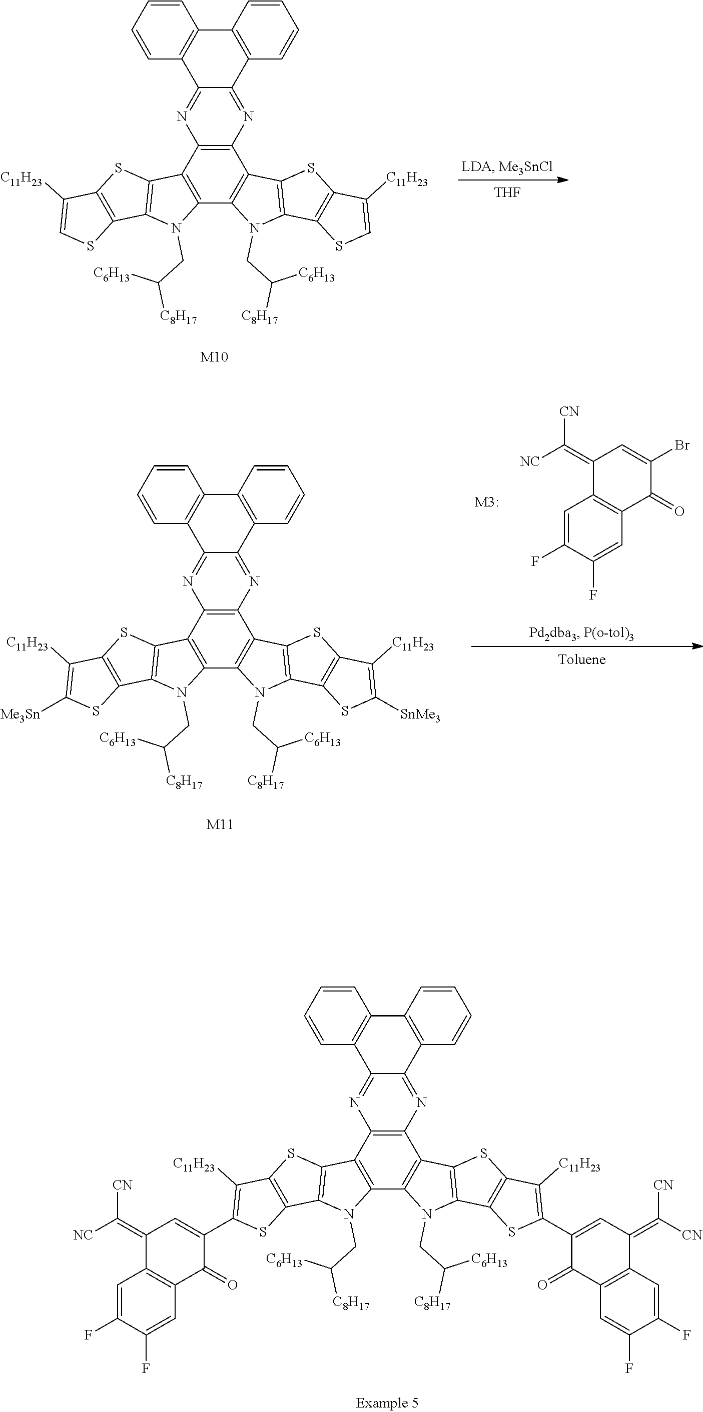

M10 (540 mg, 0.40 mmol) and anhydrous THF were added into a dried 100 mL three-neck flask. The mixture was cooled to 0° C., followed by the addition of 2.0 M LDA (1.61 mL, 3.22 mmol). The reaction was maintained below 0° C. for 30 minutes. Me₃SnCl (727 mg, 3.65 mmol, completely dissolved in anhydrous THF) was added at 0° C. After stirring at room temperature for 16 hours, water was slowly added to quench the reaction. The mixture was extracted with heptane/water three times. The organic layers were dried over magnesium sulfate, and the solvent was removed to afford M11 as an orange oil (675 mg, yield: 99%). ¹H NMR (600 MHz, CDCl₃): δ 9.76 (d, J=8.0 Hz, 2H), 8.70 (d, J=8.4 Hz, 2H), 7.93 (t, J=7.2 Hz, 2H), 7.83 (t, J=7.2 Hz, 2H), 4.71 (d, J=7.8 Hz, 4H), 2.92 (t, J=8.1 Hz, 4H), 2.27-2.17 (m, 2H), 1.96-1.90 (m, 4H), 1.54-0.72 (m, 92H), 0.66 (t, J=7.2 Hz, 6H), 0.50 (s, 18H). M11 (675 mg, 0.41 mmol) and M3 (325 mg, 1.01 mmol) were added into a 100 mL two-neck flask, followed by the addition of toluene (30 mL) and stirring with a magnetic stir bar. The mixture was degassed at room temperature for 30 minutes. Pd₂(dba)₃ (14.8 mg, 0.016 mmol) and P(o-tol)₃ (19.7 mg, 0.065 mmol) were added. The reaction mixture was heated to 90° C. and stirred for 18 hours, then cooled to room temperature. Purification by silica gel column chromatography (eluent: heptane/dichloromethane=2/1) afforded Example 5 as a black solid (240 mg, yield: 33%). ¹H NMR (500 MHz, 1,1,2,2-tetrachloroethane-d2, 100° C.): δ 9.71 (d, J=8.0 Hz, 2H), 8.77-8.72 (m, 4H), 8.19 (t, J=8.8 Hz, 2H), 8.13 (s, 2H), 7.95 (t, J=7.5 Hz, 2H), 7.89 (t, J=7.5 Hz, 2H), 4.82 (d, J=7.5 Hz, 4H), 3.27 (t, J=8.0 Hz, 4H), 2.27-2.25 (m, 2H), 2.16-2.10 (m, 4H), 1.74-1.68 (m, 4H), 1.58-1.53 (m 4H), 1.49-0.68 (m, 90H).

### Synthesis of Example 6

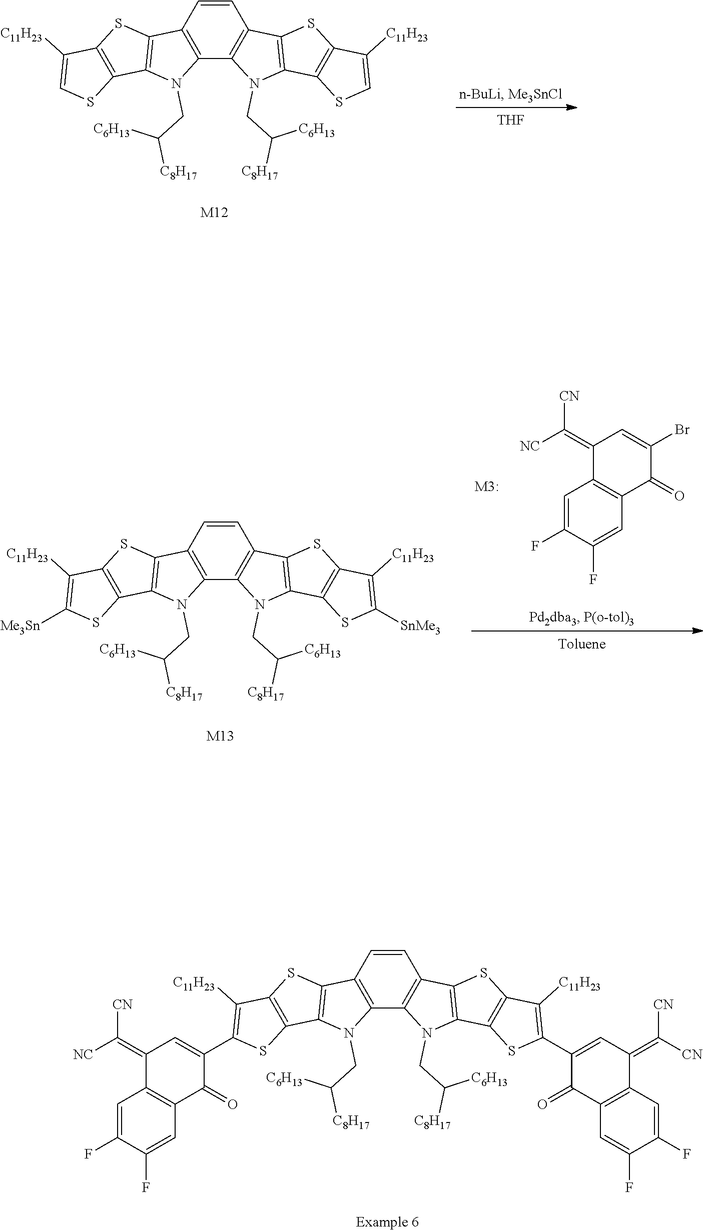

M12 (150 mg, 0.13 mmol) and anhydrous THF were added into a dried 100 mL two-neck flask. The mixture was cooled to −60° C., followed by the addition of 2.5 M n-butyllithium (n-BuLi, 0.26 mL, 0.64 mmol). The reaction was maintained below −60° C. for 1 hour. Me₃SnCl (130 mg, 0.65 mmol, completely dissolved in anhydrous THF) was added at −60° C. After stirring at room temperature for 3 hours, water was slowly added to quench the reaction. The mixture was extracted with heptane/water three times. The organic layers were dried over magnesium sulfate, and the solvent was removed to afford M13 as a bright-yellow oil (113 mg, yield: 60%). ¹H NMR (500 MHz, CDCl₃): δ 7.48 (s, 2H), 4.50 (d, J=7.5 Hz, 4H), 3.23 (t, J=6.0 Hz, 4H), 2.00-1.89 (m, 2H), 1.58-1.47 (m, 4H), 1.44-0.69 (m, 98H), 0.45 (s, 18H). M13 (110 mg, 0.075 mmol) and M3 (75 mg, 0.225 mmol) were added into a 100 mL three-neck flask, followed by the addition of toluene (7 mL). The mixture was degassed at room temperature for 30 minutes. Pd₂(dba)₃ (2.8 mg, 0.003 mmol) and P(o-tol)₃ (3.7 mg, 0.012 mmol) were added. The reaction mixture was heated to 110° C. and stirred for 16 hours, then cooled to room temperature. Purification by silica gel column chromatography (eluent: heptane/dichloromethane=2/1) afforded Example 6 as a black solid (28 mg, yield: 23%). ¹H NMR (500 MHz, CDCl₃): δ 8.77-8.73 (m, 2H), 8.19-8.16 (m, 2H), 8.01 (s, 2H), 7.54 (s, 2H), 4.57 (d, J=7.5 Hz, 4H), 3.07 (t, J=8.0 Hz, 4H), 2.33-2.30 (m, 2H), 1.98-1.95 (m, 4H), 1.43-0.70 (m, 98H).

### Synthesis of Example 7

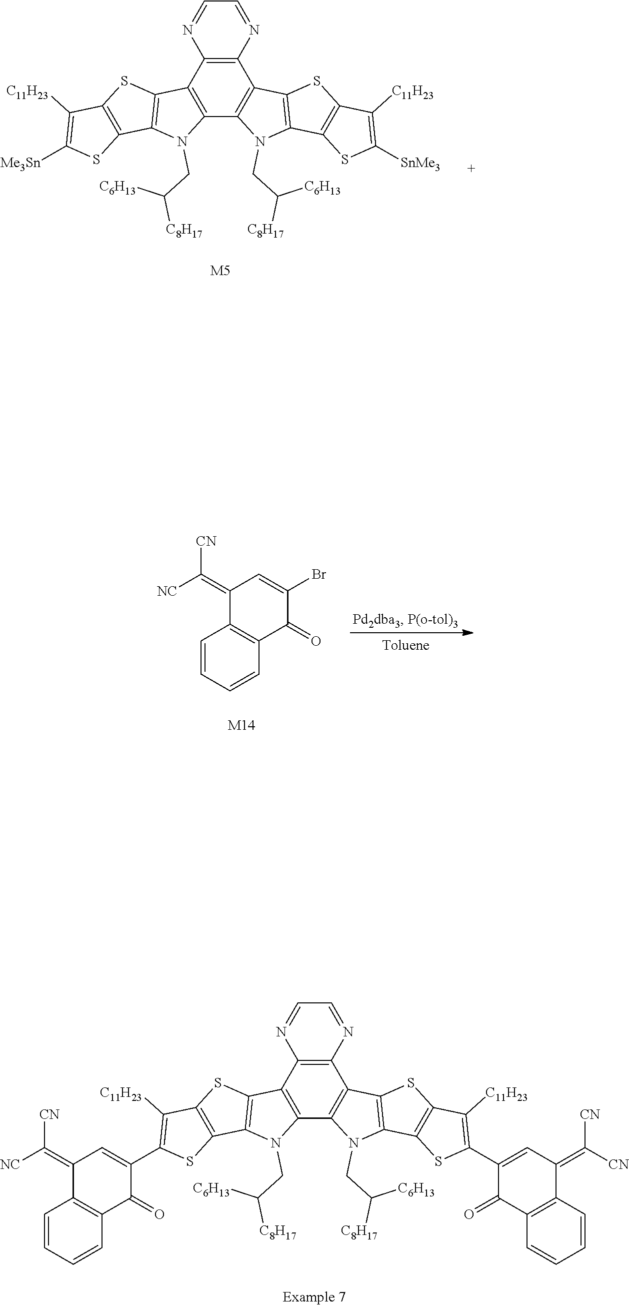

M5 (200 mg, 0.13 mmol) and M14 (94 mg, 0.33 mmol) were added into a 100 mL three-neck flask, followed by the addition of toluene (30 mL). The mixture was degassed at room temperature for 30 minutes. Pd₂(dba)₃ (5 mg, 0.0053 mmol) and P(o-tol)₃ (7 mg, 0.021 mmol) were added. The reaction mixture was heated to 90° C. and stirred for 16 hours, then cooled to room temperature. Purification by silica gel column chromatography (eluent: heptane/dichloromethane=2/1) afforded Example 7 as a black solid (84 mg, yield: 40%). ¹H NMR (500 MHz, CDCl₃): δ 8.97 (s, 2H), 8.85 (d, J=7.5 Hz, 2H), 8.39 (d, J=7.5 Hz, 2H), 8.08 (s, 2H), 7.84-7.82 (m, 4H), 4.72 (d, J=8.0 Hz, 4H), 3.12 (t, J=8.0 Hz, 4H), 2.20-2.15 (m, 2H), 2.05-2.00 (m, 4H), 1.44-1.40 (m, 4H), 1.33-1.25 (m, 26H), 1.16-0.87 (m, 52H), 0.79-0.76 (m, 10H), 0.67 (t, J=6.0 Hz, 6H).

### Synthesis of Example 8

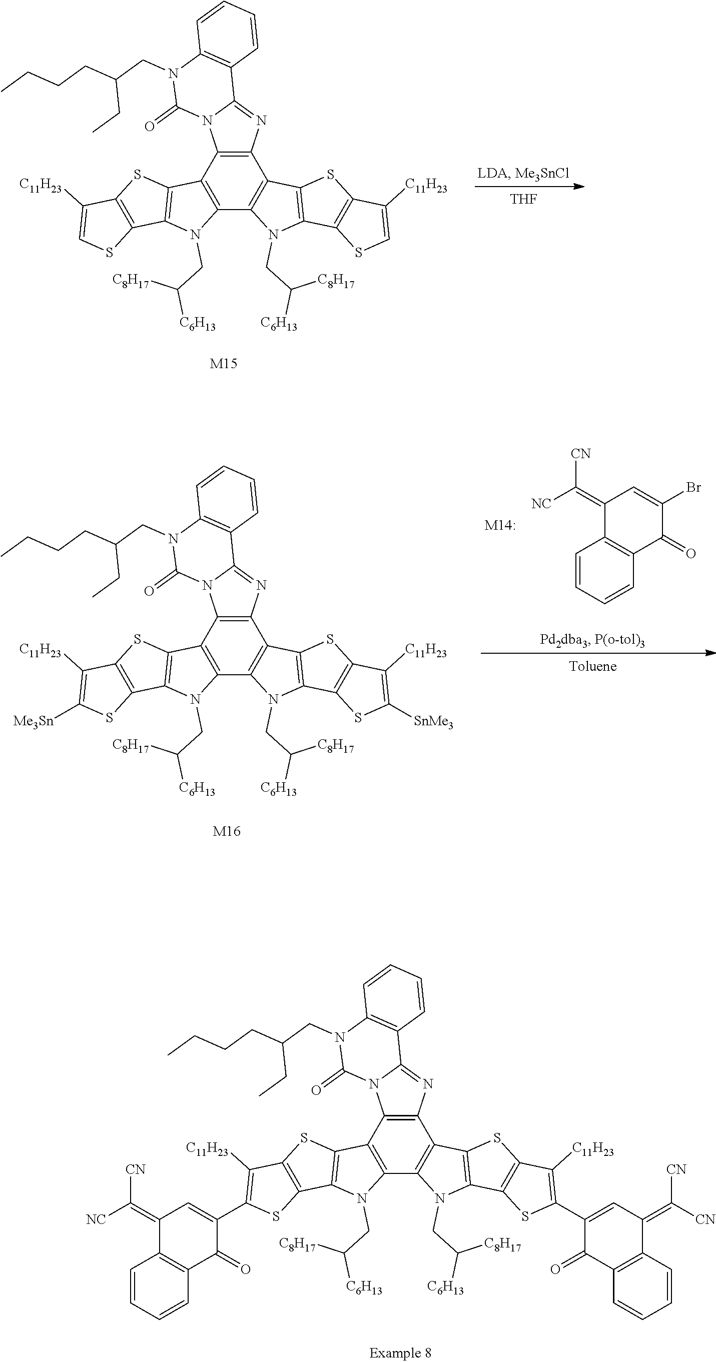

M15 (300 mg, 0.23 mmol) and anhydrous THF were added into a dried 100 mL two-neck flask. The mixture was cooled to −78° C., followed by the addition of 2.0 M LDA (0.93 mL, 1.85 mmol). The reaction was maintained below −30° C. for 1 hour. The mixture was cooled again to −78° C., and Me₃SnCl (415 mg, 2.08 mmol, completely dissolved in anhydrous THF) was added. After stirring at room temperature for 16 hours, water was slowly added to quench the reaction. The mixture was extracted with heptane/water three times. The organic layers were dried over magnesium sulfate, and the solvent was removed to afford M16 as an orange oil (310 mg, yield: 77%). ¹H NMR (600 MHz, CDCl₃): δ 8.78 (d, J=7.2 Hz, 1H), 7.59 (t, J=7.2 Hz, 1H), 7.41-7.38 (m, 2H), 4.60-4.40 (m, 6H), 2.84-2.81 (m, 4H), 2.15-2.13 (m, 2H), 1.94-1.87 (m, 5H), 1.60-0.66 (m, 112H), 0.52-0.43 (m, 18H). M16 (310 mg, 0.179 mmol) and M14 (153 mg, 0.537 mmol) were added into a 100 mL three-neck flask, followed by the addition of toluene (22 mL). The mixture was degassed at room temperature for 30 minutes. Pd₂(dba)₃ (3.3 mg, 0.0038 mmol) and P(o-tol)₃ (4.4 mg, 0.0143 mmol) were added. The reaction mixture was heated to 110° C. and stirred for 18 hours, then cooled to room temperature. Purification by silica gel column chromatography (eluent: heptane/dichloromethane=1/1) afforded Example 8 as a black solid (67 mg, yield: 20%). ¹H NMR (500 MHz, CDCl₃): δ 8.86-8.84 (m, 2H), 8.79 (d, J=7.5 Hz, 1H), 8.39-8.37 (m, 2H), 8.04 (s, 1H), 8.03 (s, 1H), 7.85-7.81 (m, 4H), 7.66 (t, J=8.0 Hz, 1H), 7.47-7.43 (m, 2H), 4.71-4.33 (m, 6H), 3.16-3.12 (m, 4H), 2.17-1.90 (m, 7H), 2.21-1.87 (m, 7H), 1.62-0.65 (m, 112H).

### Synthesis of Example 9

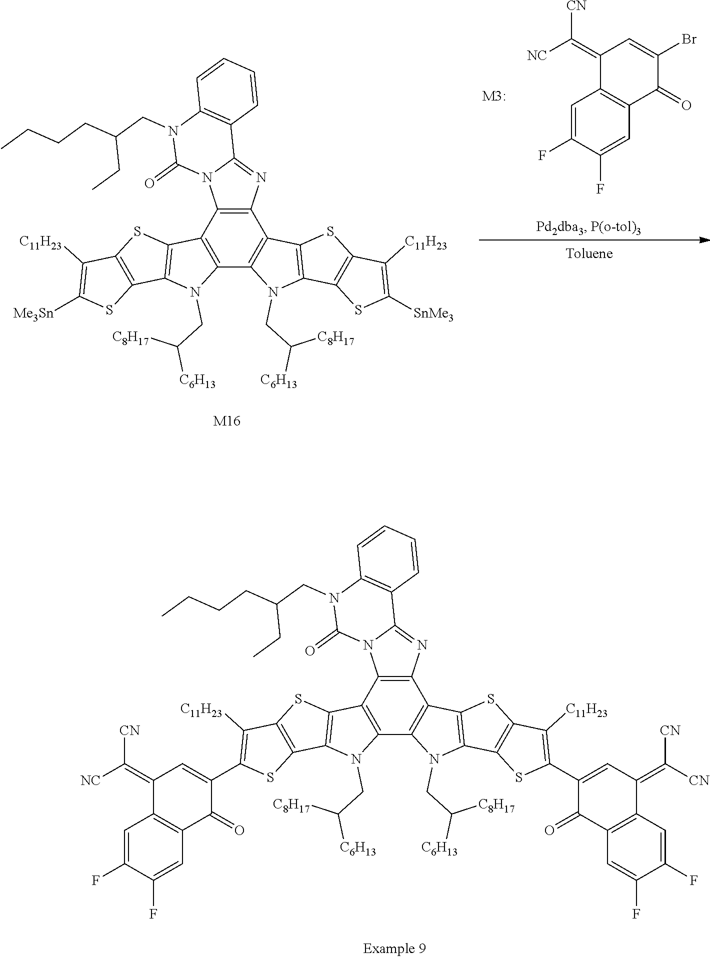

M16 (510 mg, 0.23 mmol) and M3 (290 mg, 0.69 mmol) were added into a 100 mL three-neck flask, followed by the addition of toluene (28 mL). The mixture was degassed at room temperature for 30 minutes. Pd₂(dba)₃ (10.8 mg, 0.012 mmol) and P(o-tol)₃ (14.3 mg, 0.047 mmol) were then added. The reaction mixture was heated to 110° C. and stirred for 18 hours, and subsequently cooled to room temperature. Purification by silica gel column chromatography (eluent: heptane/dichloromethane=1/1) afforded Example 9 as a black solid (107 mg, yield: 20%). ¹H NMR (500 MHz, CDCl₃): δ 8.80-8.73 (m, 3H), 8.20-8.16 (m, 2H), 8.04 (s, 1H), 8.03 (s, 1H), 7.67 (t, J=8.0 Hz, 1H), 7.48-7.43 (m, 2H), 4.68-4.32 (m, 6H), 3.16-3.12 (m, 4H), 2.17-1.90 (m, 7H), 2.17-1.87 (m, 7H), 1.62-0.65 (m, 112H).

Material testing of organic semiconductor material Example 1 to Example 9 and Comparative Example Y-QC4F includes material optical property testing:

The structure of Comparative Example Y-QC4F is as follows: (Adv. Mater. 2024, 2406950)

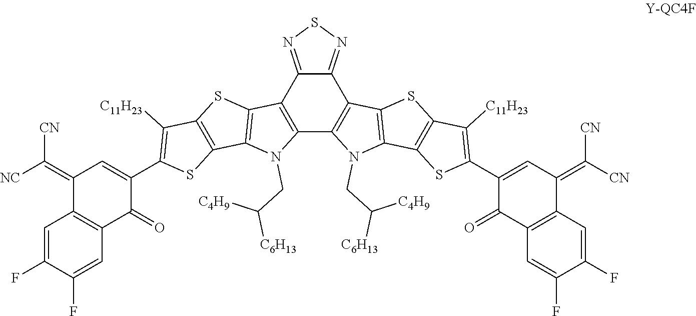

The difference between Comparative Example Y-QC4F and the organic semiconductor materials of the present invention lies in the structural unit represented by Formula I. In Comparative Example Y-QC4F, A is composed of one six-membered ring and one five-membered heterocyclic ring, whereas in the present invention, A in Formula I is selected from a monocyclic ring or a polycyclic ring comprising at least two six-membered rings. In other words, the structural unit A of Formula I in the present invention excludes the substituent group of Comparative Example Y-QC4F.

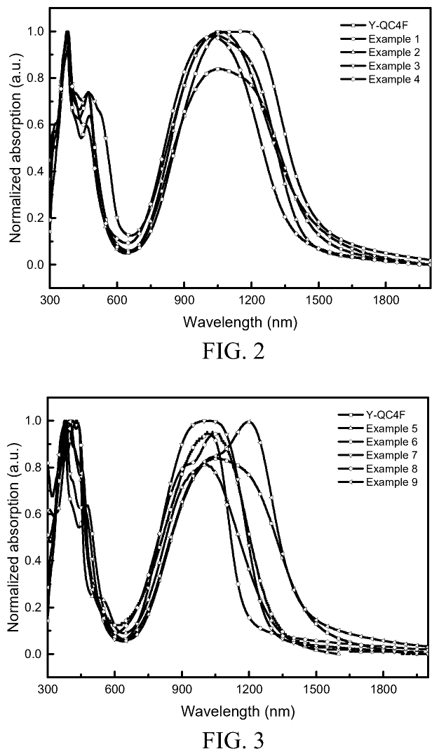

Please refer to FIG. 2, FIG. 3, and Table 1. FIG. 2 shows absorption spectra in thin-film state of Comparative Example Y-QC4F and Example 1 to Example 4 of organic semiconductor materials. FIG. 3 shows absorption spectra in thin-film state of Comparative Example Y-QC4F and Example 5 to Example 9 of organic semiconductor materials. Table 1 shows material tests of the organic semiconductor materials of Comparative Example Y-QC4F and Example 1 to Example 9 (including the data shown in FIG. 2 and FIG. 3)

TABLE 1
Material tests of the organic semiconductor materials
of Comparative Example Y-QC4F and Example 1 to Example
9 (including the data shown in FIG. 2 and FIG. 3).

| Organic | | | | | |
|---|---|---|---|---|---|
| semiconductor | λₛₒₗₙ^{max} | λ_{film}^{max} | E_{g}^{opt} | HOMO | LUMO |
| materials | (nm) | (nm) | (eV) | (eV) | (eV) |
| Example 1 | 941 | 1181 | 0.85 | −5.48 | −4.63 |
| Example 2 | 938 | 1043 | 0.88 | −5.45 | −4.57 |
| Example 3 | 964 | 1064 | 0.85 | −5.40 | −4.55 |
| Example 4 | 934 | 1040 | 0.91 | −5.37 | −4.46 |
| Example 5 | 976 | 1015 | 0.97 | −5.47 | −4.50 |
| Example 6 | 961 | 1068 | 0.95 | −5.44 | −4.49 |
| Example 7 | 888 | 1029 | 0.98 | −5.41 | −4.43 |
| Example 8 | 938 | 1036 | 1.05 | −5.44 | −4.39 |
| Example 9 | 1001 | 1195 | 0.87 | −5.33 | −4.46 |
| Y-QC4F | 912 | 1062 | 0.84 | −5.48 | −4.64 |

As shown in Table 1, the organic semiconductor materials of Example 1 to Example 9 of the present invention, as well as Comparative Example Y-QC4F, exhibit maximum absorption peaks in thin-film state at wavelengths greater than 1000 nm, and all of their optical bandgaps are less than 1.15 eV. As shown in FIG. 2 and FIG. 3, the thin-film absorption spectra of the organic semiconductor materials demonstrate strong absorption properties in the range of 600-1600 nm. Accordingly, the applicable spectral range extends from the visible region to the infrared region, enabling further application in short-wave infrared organic photodetectors.

Thermal stability performance test of single material absorbance:

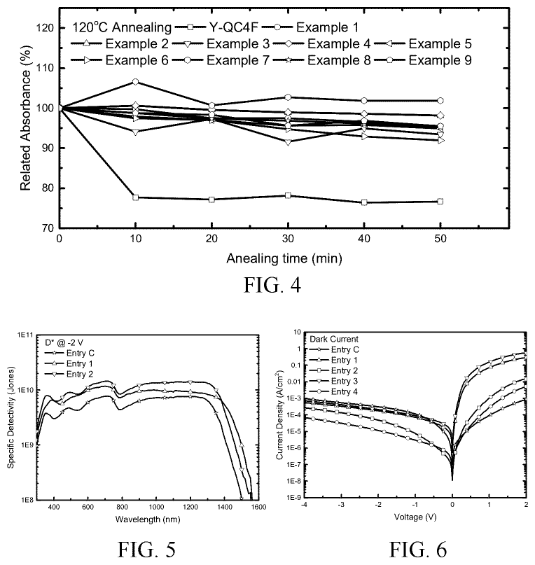

Please refer to FIG. 4 and Table 2. FIG. 4 shows thermal stability variations of Comparative Example Y-QC4F and Example 1 to Example 9 of organic semiconductor materials under a baking condition of 120° C. Table 2 shows the thermal stability tests of the organic semiconductor materials of Comparative Example Y-QC4F and Example 1 to Example 9 (including the data corresponding to FIG. 4).

TABLE 2
Thermal stability tests of the organic semiconductor materials
of Comparative Example Y-QC4F and Example 1 to Example 9.

| Organic | Baking condition of 120° C. | | | | | |
|---|---|---|---|---|---|---|
| semiconductor | Origin | 10 | 20 | 30 | 40 | 50 |
| materials | (0 min) | min | min | min | min | min |
| Y-QC4F | 100.0 | 77.6 | 77.1 | 78.1 | 76.4 | 76.6 |
| Example 1 | 100.0 | 99.8 | 97.4 | 97.4 | 96.6 | 95.5 |
| Example 2 | 100.0 | 100.6 | 99.6 | 99.0 | 98.6 | 98.1 |
| Example 3 | 100.0 | 94.1 | 97.3 | 91.6 | 94.9 | 93.5 |
| Example 4 | 100.0 | 100.6 | 99.6 | 99.0 | 98.6 | 98.1 |
| Example 5 | 100.0 | 97.9 | 97.0 | 95.6 | 95.8 | 94.9 |
| Example 6 | 100.0 | 97.5 | 97.0 | 94.7 | 92.9 | 91.9 |
| Example 7 | 100.0 | 106.6 | 100.7 | 102.7 | 101.9 | 101.9 |
| Example 8 | 100.0 | 98.8 | 97.4 | 96.8 | 96.1 | 95.3 |
| Example 9 | 100.0 | 98.8 | 98.4 | 95.6 | 96.9 | 95.5 |

To evaluate the thermal stability of the materials after heating, thin-film organic semiconductor materials were baked at 120° C. in air for up to 50 minutes. During the baking process, the absorbance at 1100 nm was measured at 0, 10, 20, 30, 40, and 50 minutes, respectively. FIG. 4 and Table 2 present the results normalized to the absorbance at 0 minutes, which was set as 100%, and the absorbance change was analyzed every 10 minutes during baking. As shown in FIG. 4 and Table 2, the absorbance of Comparative Example Y-QC4F continuously decreased after heating and dropped to 76%, corresponding to a 24% reduction. In contrast, the organic semiconductor materials of Example 1 to Example 9 of the present invention retained 91% to 107% of their absorbance after baking, corresponding to an absorbance variation within +9%. Comparatively, the degree of change observed in Comparative Example Y-QC4F is more than 2.5 times that of Example 1 to Example 9 of the present invention, demonstrating that Example 1 to Example 9 exhibit superior thermal stability.

Preparation and performance testing of organic photodetectors of organic optoelectronic devices:

A glass coated by a pre-patterned indium tin oxides (ITO) with a sheet resistance of ˜15Ω/sq is used as a substrate. The substrate is ultrasonically oscillated in soap deionized water, deionized water, acetone, and isopropanol in sequence, and washed in each step for 15 minutes. The washed substrate is further treated with a UV-ozone cleaner for 15 minutes. The top coat of AZO (Aluminum doped zinc oxide nanoparticle) solution is spin coated on the ITO substrate with a spin rate of 2000 rpm for 40 seconds, and then baked at 120° C. in air for 5 minutes to form an electron transporting layer (ETL). The active layer solution comprises the aforementioned organic composition, wherein at least one P-type organic semiconductor material is used as a donor material, and at least one N-type organic semiconductor material (i.e., the organic semiconductor material of the present invention) is used as an acceptor material (the weight ratio of donor material to acceptor material is in the range of 1:0.5-4). The concentration of the donor material was 5˜20 mg/mL. In order to completely dissolve the active layer material, the active layer solution needs to be stirred on a hot plate at 100° C. for at least 3 hours. After completely dissolving the active layer material, the active layer solution is filtered with PTFE filter membrane (pore size 0.45˜1.2 μm) and heated for 1 hour. Then, the active layer solution is cooled to the room temperature for spin coating, and the spin rate was used to control the film thickness in the range of 80-800 nm. Finally, the thin film formed by the coated active layer is annealed at 100° C. for 5 minutes, and then transferred to a thermal evaporation machine. A thin layer (8 nm) of MoO₃ is deposited as a hole transporting layer (HTL) under a vacuum of 3×10⁻⁶ Torr. In this experiment, a Keithley™ 2400 source meter was used to record the dark current density (J_{dark}, at a bias of 0˜−8 V) in the absence of light. External quantum efficiency system was used to measure external quantum efficiency (EQE) with a range of 300-1800 nm (bias voltage 0˜−4 V), and silicon (300-1100 nm) and germanium (600-1800 nm) are used for light source calibration.

It should be noted here that, in practical applications, the first electrode preferably has good light transmittance. The first electrode is usually made of the transparent conductive material, preferably selected from one of the following conductive material groups: indium oxide, tin oxide, fluorine-doped tin oxide (FTO) derivative, or composite metal oxides such as indium tin oxide (ITO) and indium zinc oxide (IZO). The material of the second electrode is a conductive metal, preferably silver or aluminum, more preferably silver. Suitable and preferred materials for electron transporting layer include, but are not limited to, metal oxides such as ZnOₓ, aluminum doped ZnO (AZO), TiOₓ or nanoparticles thereof, salts (such as LiF, NaF, CsF, Cs₂CO₃), amines (such as primary amines, secondary or tertiary amines), conjugated polymer electrolytes (such as polyethyleneimine), conjugated polymers (such as poly[3-(6-trimethylammoniumhexyl)thiophene], poly(9,9)-bis(2-ethylhexyl-fluorene)-b-poly[3-(6-trimethylammoniumhexyl)thiophene] or poly[(9,9-bis(3′-(N,N-dimethylamino))propyl)-2,7-fluorene)-alt-2,7-(9,9-dioctylfluorene)], and organic compounds such as tris(8-quinolinyl)-aluminum (III) (Alq₃), 4,7-diphenyl-1,10-phenanthroline, or a combination of one or more of the foregoing. Suitable and preferred materials for hole transporting layer include, but are not limited to metal oxides such as ZTO (Zinc Tin Oxide), MoOₓ, WOₓ, NiOₓ, SnOₓ and nanoparticles thereof, metal-containing salts, such as copper sulfide, copper thiocyanate, copper iodide, copper indium sulfide, lead sulfide, cobalt acetate, and tungsten disulfide, conjugated polymer electrolytes such as PEDOT:PSS, polymeric acids such as polyacrylates, conjugated polymers such as polytriarylamine (PTAA), insulating polymers such as Nafion films, polyethyleneimine and polystyrene sulfonates, organic compounds such as N,N′-diphenyl-N,N′-bis(1-naphthyl) (1,1′-biphenyl)-4,4′-diamine (NPB), N,N′-diphenyl-N,N′-(3-methylbenzene base)-1,1′-biphenyl-4,4′-diamine (TPD), and a combination of one or more of the above.

Subsequently, the organic semiconductor materials of Example 1 to Example 3 and Comparative Example Y-QC4F are prepared to organic optoelectronic devices corresponding to Entry 1 to Entry 4 and Comparative Entry C, for device performance evaluation and comparison. Please refer to Table 3. Table 3 shows the formulations of Comparative Entry C and Entry 1 to Entry 4 of the organic optoelectronic devices. Among these, although Entry 1 and Entry 2 both use D28 in combination with Example 1, the difference lies in the processing solvent. Since Comparative Entry C is processed using the halogenated solvent chlorobenzene, and although the organic semiconductor materials of the present invention are soluble in the non-halogenated solvent o-xylene, Entry 1 (using chlorobenzene) and Entry 2 (using o-xylene) were designed to eliminate solvent-related influences, thereby ensuring a more complete and reliable comparison of device performance.

TABLE 3
Formulations of Comparative Entry C and Entry 1
to Entry 4 of the organic optoelectronic devices.

| Formulations | | | |
|---|---|---|---|
| of the organic | | | |
| optoelectronic | | | |
| devices | P-type | N-type | Process solvent |
| Entry C | D28 | Y-QC4F | Chlorobenzene |
| Entry 1 | D28 | Example 1 | Chlorobenzene |
| Entry 2 | D28 | Example 1 | o-Xylene |
| Entry 3 | D28 | Example 2 | o-Xylene |
| Entry 4 | D28 | Example 3 | o-Xylene |

Please refer to FIG. 5, FIG. 6, and Table 4. FIG. 5 shows detectivity versus wavelength characteristics of Comparative Entry C, Entry 1 and Entry 2 of organic optoelectronic devices under a bias voltage of −2 V. FIG. 6 shows dark current density-voltage characteristics of Comparative Entry C and Entry 1 to Entry 4 of organic optoelectronic devices under different bias voltages. Table 4 shows the comparison of dark current density and detectivity for Comparative Entry C, Entry 1 and Entry 2 of the organic optoelectronic devices.

TABLE 4
Comparison of dark current density and detectivity for Comparative
Entry C, Entry 1 and Entry 2 of the organic optoelectronic devices.

| Organic | | Dark | | |
|---|---|---|---|---|
| optoelectronic | J_{d} @-2 V | Current | D* @-2 V | Detectivity |
| device | (A/cm²) | Difference | (Jones) | Difference |
| Entry C | 8.56 × 10⁻⁴ | STD | 6.38 × 10⁹ | STD |
| Entry 1 | 1.77 × 10⁻⁴ | decreased | 8.31 × 10⁹ | increased |
| | | by 4.8× | | by 1.3× |
| Entry 2 | 2.17 × 10⁻⁴ | decreased | 1.33 × 10¹⁰ | increased |
| | | by 3.9× | | by 2.1× |

As shown in FIG. 5, FIG. 6, and Table 4, when the result measured by Entry C is used as the standard value, Entry 1 exhibits a dark current density that is 4.8 times lower than that of Entry C, and a detectivity that is 1.3 times higher than that of Entry C. This indicates that the organic optoelectronic device (Entry 1) fabricated using the organic semiconductor material of Example 1 of the present invention demonstrates excellent dark current density and detectivity under the same fabrication conditions as Entry C. Furthermore, when the result measured by Entry C is used as the standard value, Entry 2 exhibits a dark current density that is 3.9 times lower than that of Entry C, and a detectivity that is 2.1 times higher than that of Entry C. This demonstrates that even when the organic optoelectronic device (Entry 2) fabricated using the organic semiconductor material of Example 1 of the present invention is processed with a non-halogenated solvent, Entry 2 still exhibits excellent dark current density and detectivity performance. Accordingly, the organic optoelectronic devices fabricated from the organic semiconductor materials of the present invention not only exhibit excellent detectivity, but also achieve superior performance when processed using non-halogenated solvents.

As described above, please further refer to Table 5. Table 5 shows a comparison of the dark current densities of Comparative Entry C and Entry 1 to Entry 4 of the organic optoelectronic devices.

TABLE 5
Comparison of the dark current densities of Comparative Entry
C and Entry 1 to Entry 4 of the organic optoelectronic devices.

| Organic | | |
|---|---|---|
| optoelectronic | J_{d} @-2 V | Dark Current |
| device | (A/cm²) | Difference |
| Entry C | 8.56 × 10⁻⁴ | STD |
| Entry 1 | 1.77 × 10⁻⁴ | decreased by 4.8× |
| Entry 2 | 2.17 × 10⁻⁴ | decreased by 3.9× |
| Entry 3 | 1.14 × 10⁻⁵ | decreased by 75.1× |
| Entry 4 | 4.79 × 10⁻⁵ | decreased by 17.9× |

As shown in FIG. 6 and Table 5, the organic optoelectronic devices of Entry 1 to Entry 4 exhibit lower dark current densities under a bias voltage of −2 V compared to Comparative Entry C, with reductions reaching as much as 75.1-fold. These results clearly demonstrate that the organic optoelectronic devices fabricated under non-halogenated processing conditions exhibit significantly improved performance, with particularly notable enhancements in the reduction of dark current.

In summary, the organic semiconductor materials of the present invention exhibit optical bandgaps below 1.15 eV and possess excellent solubility and thermal stability. In addition, the organic optoelectronic devices of the present invention are organic photodetectors applicable to short-wave infrared light, and are fabricated using non-halogenated solvents, which are more environmentally friendly than commonly used halogenated solvents such as chloroform or chlorobenzene reported in the literature. The organic optoelectronic devices of the present invention exhibit low dark current densities, good photoresponse and detectivity in the short-wave infrared region, and excellent thermal stability.

With the detailed description of the above embodiments, it is hoped that the features and spirit of the present invention can be more clearly described, and the scoped of the present invention is not limited by the embodiments disclosed above. On the contrary, the intention is to cover various changes and equivalent arrangements within the scope of the patents to be applied for in the present invention.

What is claimed is:

1. An organic semiconductor material comprising a structure of Formula I: 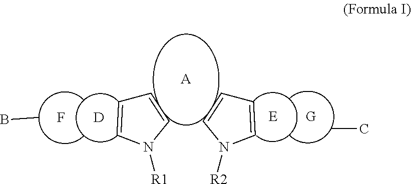
wherein A is a monocyclic ring or polycyclic ring, and the polycyclic ring comprises at least two six-membered rings;
B and C are polycyclic rings, each comprising at least one six-membered ring;
D, E, F, and G are monocyclic rings or polycyclic rings, each comprising at least one five-membered ring; and
R1 and R2 are independently selected from the following groups and any combinations thereof: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl.

2. The organic semiconductor material of claim 1, wherein A is a C6-C30 monocyclic ring or polycyclic ring, and the polycyclic ring comprises at least two six-membered rings; B and C are C3-C20 aryl with substituents or heteroaryl with substituents, wherein the substituents comprise at least one ketone group; and D, E, F, and G are a C4-C30 monocyclic ring or polycyclic ring.

3. The organic semiconductor material of claim 1, wherein an optical bandgap of the organic semiconductor material is less than 1.15 eV.

4. The organic semiconductor material of claim 1, wherein A is selected from one of the following structures, in which the dashed line represents an outward bonding position: 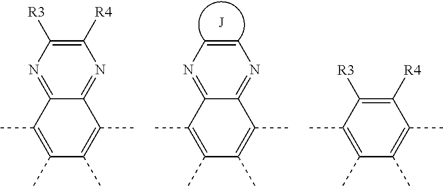
wherein R3 and R4 are the same or different and are independently selected from the following groups and any combinations thereof: hydrogen, deuterium, tritium, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl; and
J is selected from the following structures, in which the dashed line represents an outward bonding position: 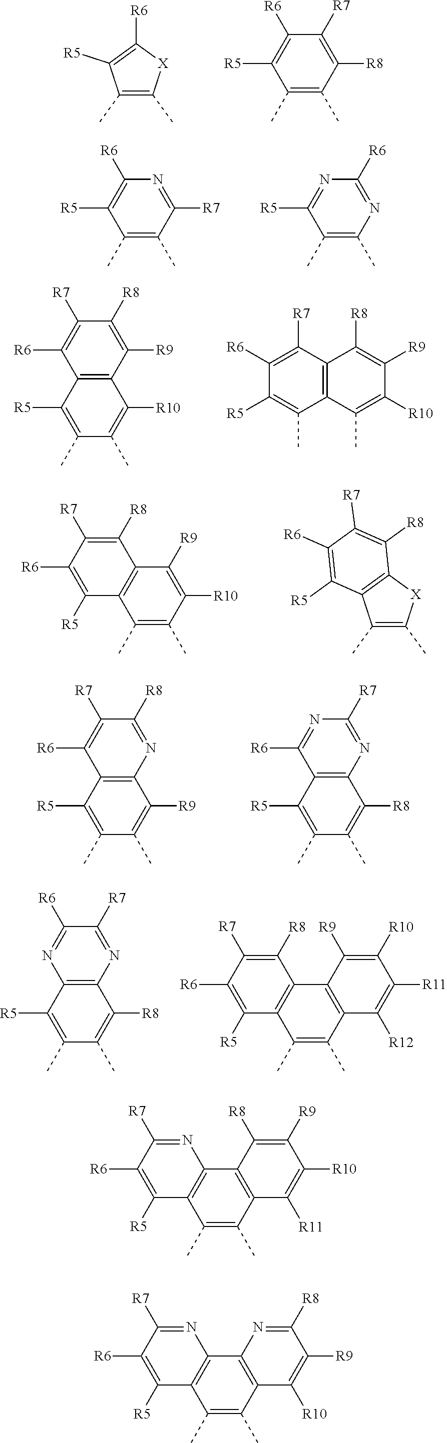 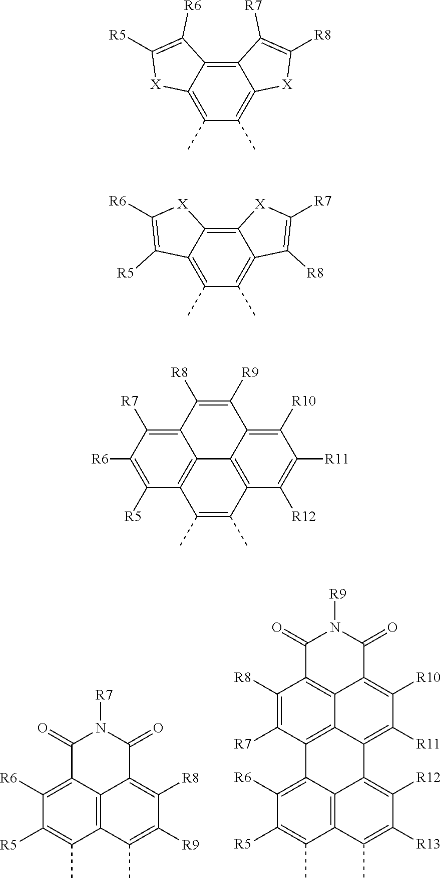
wherein X is selected from O, S, Se, or N—R14; and
R5, R6, R7, R8, R9, R10, R11, R12, R13, and R14 are independently selected from the following groups and any combinations thereof: hydrogen, deuterium, tritium, halogen, cyano, C1-C30 alkyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl.

5. The organic semiconductor material of claim 1, wherein B and C are independently selected from the following structures, in which the dashed line represents an outward bonding position: 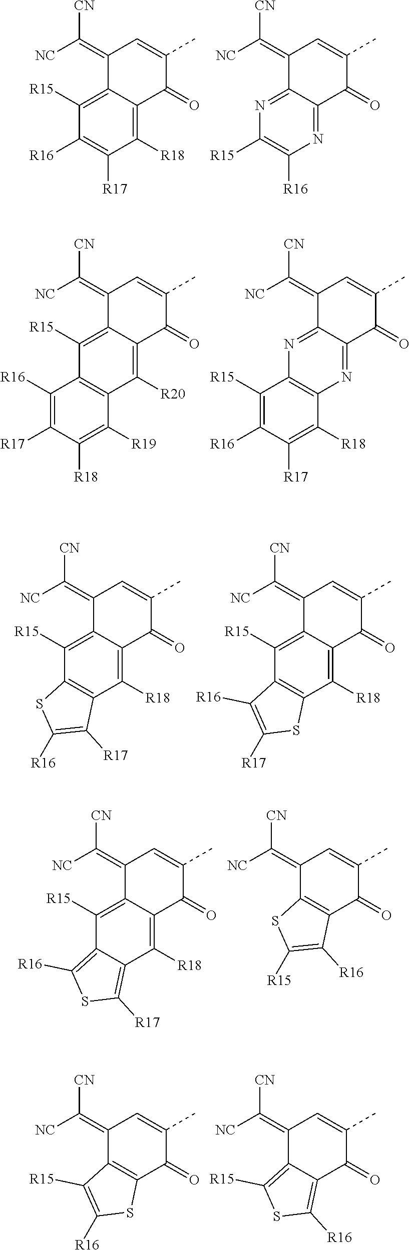 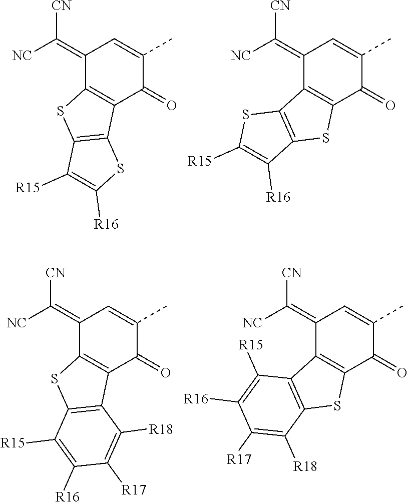
wherein R15, R16, R17, R18, R19, and R20 are independently selected from the following groups and any combinations thereof: C1-C30 alkyl, C1-C30 silyl, C1-C30 alkoxy, C1-C30 alkylthio, C1-C30 haloalkyl, halogen, hydrogen, deuterium, tritium, and cyano.

6. The organic semiconductor material of claim 1, wherein D and E are independently selected from the following structures, in which the dashed line represents an outward bonding position: 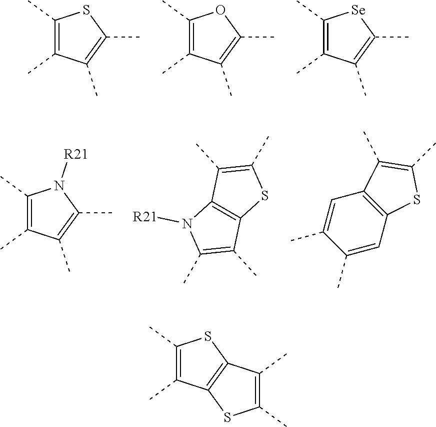
wherein R21 is selected from the following groups and any combinations thereof: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl.

7. The organic semiconductor material of claim 1, wherein F and G are independently selected from the following structures, in which the dashed line represents an outward bonding position: 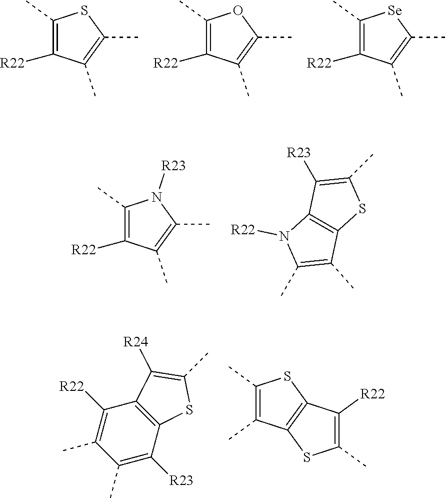
wherein R22, R23, and R24 are independently selected from the following groups and any combinations thereof: C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, C2-C30 esteryl heteroaryl, and hydrogen.

8. An organic composition, comprising:
at least one P-type organic semiconductor material, which comprises at least one organic conjugated polymer or one organic conjugated small molecule; and
at least one N-type organic semiconductor material, which comprises at least one organic semiconductor material of claim 1.

9. The organic composition of claim 8, wherein the P-type organic semiconductor material is further selected from at least one organic conjugated polymer, and the organic conjugated polymer is formed from a plurality of monomer units, the monomer units being selected from the following structures and combinations thereof: 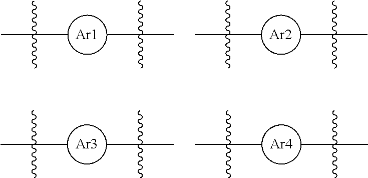
wherein Ar1, Ar2, Ar3, and Ar4 are independently selected from a monocyclic ring or a polycyclic ring.

10. The organic composition of claim 9, wherein the organic conjugated polymer further comprises the following structures and any combinations thereof: 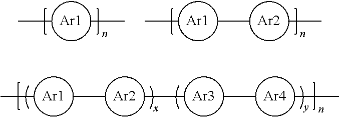
wherein Ar1, Ar2, Ar3, and Ar4 are monocyclic rings or polycyclic rings each comprising C3-C30 ring atoms;
n is the number of repeating units, and n is a positive integer from 1 to 1000; and
x and y are mole fractions, wherein 0<x<1, 0<y<1, and x+y=1.

11. The organic composition of claim 10, wherein Ar1 and Ar3 are independently selected from the following structures: 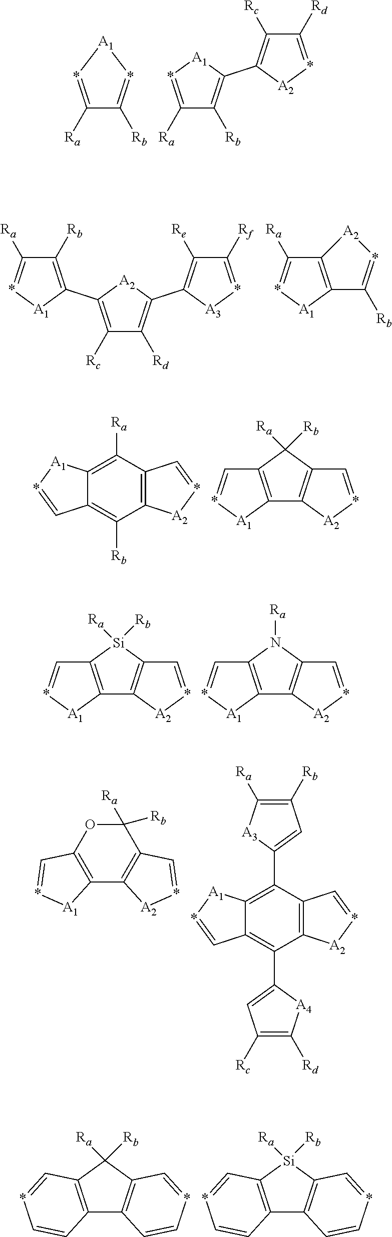 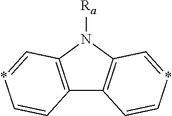
wherein A₁, A₂, A₃, and A₄ are independently selected from O, S, and Se;
Rₐ, R_{b}, R_{c}, R_{d}, Rₑ, and R_{f} are independently selected from the following groups and any combinations thereof: hydrogen, halogen, cyano, C1-C30 alkyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl; and
* represents a single bond outward bonding.

12. The organic composition of claim 10, wherein Ar2 and Ar4 are independently selected from the following structures: 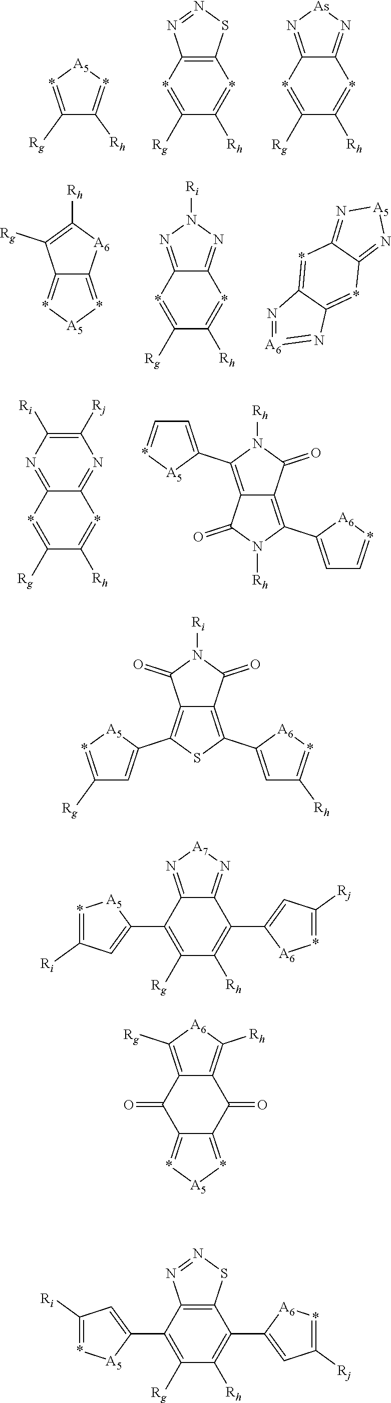 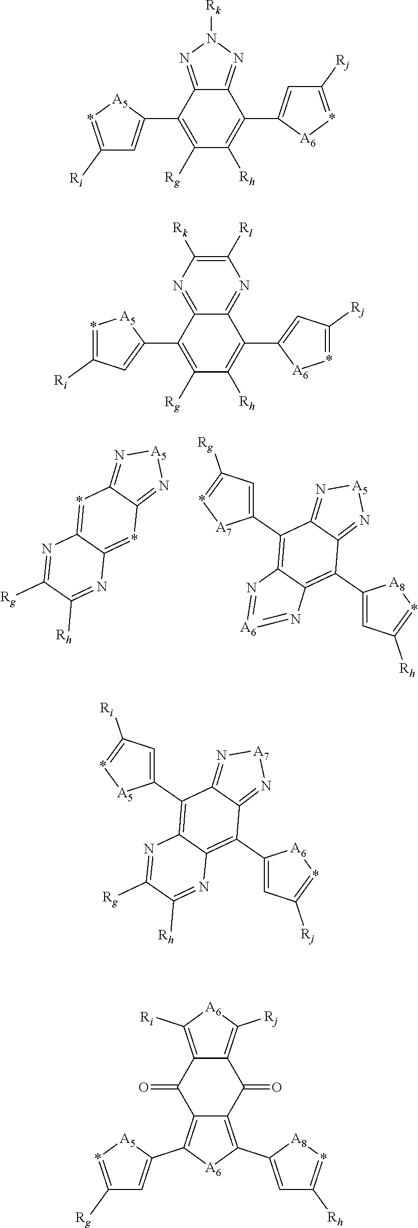 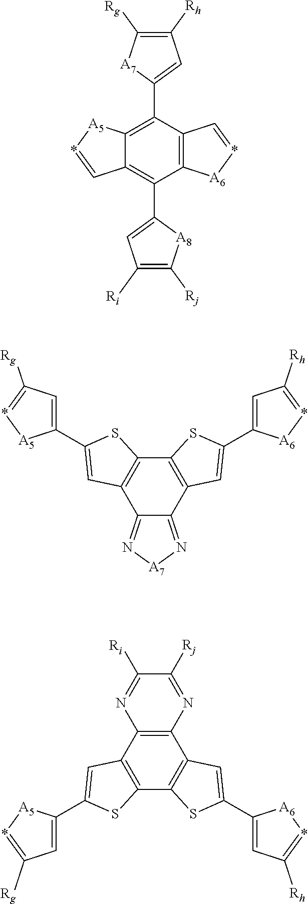
wherein A₅, A₆, A₇, and A₈ are independently selected from O, S, and Se;
R_{g}, Rₕ, Rᵢ, Rⱼ, Rₖ, and Rₗ are independently selected from the following groups and any combinations thereof: hydrogen, halogen, cyano, C1-C30 alkyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthio heteroaryl, C1-C30 haloalkyl aryl, C1-C30 haloalkyl heteroaryl, C2-C30 esteryl aryl, and C2-C30 esteryl heteroaryl; and
* represents a single bond outward bonding.

13. An organic optoelectronic device comprising:
a first electrode;
an active layer which at least comprises the organic semiconductor material of claim 1; and
a second electrode, wherein the active layer is disposed between the first electrode and the second electrode, and at least one of the first electrode and the second electrode is a transparent or semi-transparent electrode.

14. An organic optoelectronic device comprising:
a first electrode;
an active layer which at least comprises the organic composition of claim 8; and
a second electrode, wherein the active layer is disposed between the first electrode and the second electrode, and at least one of the first electrode and the second electrode is a transparent or semi-transparent electrode.
